(19) United States
(12) Patent Application Publication
(10) Pub. No.: US 2023/0347100 A1
(43) Pub. Date: Nov. 2, 2023

(54) ARTIFICIAL INTELLIGENCE-GUIDED VISUAL NEUROMODULATION FOR THERAPEUTIC OR PERFORMANCE-ENHANCING EFFECTS

(71) Applicant: DANDELION SCIENCE CORP., New York, NY (US)

(72) Inventors: Adam HANINA, New York, NY (US); Ekaterina MALAKHOVA, Highlands Mills, NY (US); Dan NEMRODOV, Jersey City, NJ (US)

(21) Appl. No.: 18/044,054

(22) PCT Filed: Sep. 3, 2021
(86) PCT No.: PCT/US2021/049080

§ 371 Date: Mar. 3, 2023

Related U.S. Application Data
(60) Provisional application No. 63/074,150, filed on Sep. 3, 2020.
(60) Provisional application No. 63/076,247, filed on Sep. 9, 2020.
(60) Provisional application No. 63/087,579, filed on Oct. 5, 2020.

(51) Int. Cl. A61M 21/00

(52) U.S. Cl. CPC A61M 21/00; A61M 2021/005

(57) ABSTRACT

Systems and methods to generate non-figurative visual neuromodulatory codes adapted to produce physiological responses having therapeutic or performance-enhancing effects. A code is rendered based on a set of rendering parameters and output to be viewed simultaneously by a plurality of subjects. Physiological responses of each of the subjects are measured during the outputting. A value of an outcome function is calculated based on the physiological responses. An updated predictive model is determined based on a current predictive model and the calculated value of the outcome function. The predictive model provides an estimated value of the outcome function for a given set of rendering parameters. Values are calculated for a set of adapted rendering parameters. The method is iteratively repeated using the set of adapted rendering parameters to produce an adapted visual neuromodulatory code, until a defined set of stopping criteria are satisfied.

### CROSS-REFERENCE TO RELATED APPLICATIONS

This application claims priority to U.S. Provisional Patent Appln. No. 63/074,150 (filed Sep. 3, 2020), U.S. Provisional Patent Appln. No. 63/076,247 (filed Sep. 9, 2020), and U.S. Provisional Patent Appln. No. 63/087,579 (filed Oct. 5, 2020). The entire content of all of these applications is incorporated herein by reference.

### BACKGROUND

### Technical Field

The present disclosure generally relates to generating and delivering visual neuromodulatory codes to produce neurological and physiological responses having therapeutic or performance-enhancing effects.

### Description of the Related Art

The field of computational neuroscience has yielded approaches to understanding and interacting with the brain. Hard-wired brain-machine interfaces and brain-computer interfaces have yielded promising results in both “reading” and “writing” to the brain, but they are expensive, difficult to scale, and can be highly invasive.

It has been shown that visual neurons respond preferentially to some stimuli over others. This discovery has led to the study of neural coding, which is a neuroscience field concerned with characterizing the relationship between a stimulus and neuronal responses. The link between stimulus and response can be studied from two opposite points of view. Neural encoding provides a map from stimulus to response, which helps in understanding how neurons respond to a wide variety of stimuli and in constructing models that attempt to predict responses to other stimuli. Neural decoding provides a reverse map, from response to stimulus, to help in reconstructing a stimulus, or certain aspects of that stimulus, from the spike sequences it evokes.

Neurons in the visual cortex fire action potentials when visual stimuli, e.g., images, appear within their receptive field. By definition, the receptive field is the region within the entire visual field that elicits an action potential. But, for any given neuron, it may respond best to a subset of stimuli within its receptive field. This property is called neuronal tuning. In the earlier visual areas, neurons have simpler tuning. For example, a neuron in V1 may fire to any vertical stimulus in its receptive field. In the higher visual areas, neurons have complex tuning. For example, in the inferior temporal cortex (IT), a neuron may fire only when a certain face appears in its receptive field.

A challenge in delineating neuronal tuning in the visual cortex is the difficulty of selecting particular stimuli from the vast set of all possible stimuli. Using natural images reduces the problem, but it is impossible to present a neuron with all possible natural stimuli. Conventionally, investigators have used hand-picked stimuli based on hypotheses that particular cortical areas encode specific visual features. Despite some success with hand-picked stimuli, the field might have missed stimulus properties that better reflect the potential of tuning of cortical neurons.

Sensory processing plays a role in most theoretical explanations of emotion, yet conventional neuroscientific views consider emotion to be driven by specialized brain regions. Activity in the sensory cortex (e.g., visual areas V1 to V4 of the visual cortex) is traditionally thought to be antecedent to emotion. Emotions can be evoked exogenously or by stimuli coming from other senses. However, a relationship between visual sensory processing, in particular, and emotion has not been fully explored, nor has there been an examination of the relationship between visual sensory processing and neurological responses having therapeutic effects.

Researchers have used deep artificial neural network ventral stream models to synthesize new patterns of luminous power to control the neural firing activity of particular selected neural sites in cortical visual area V4 of monkeys. This work included using synthesized images to stretch the maximal firing rate of any single targeted neural site beyond its naturally occurring maximal rate and to independently control every neural site in a small recorded population so that one neural site is pushed to be highly active while all other nearby sites are simultaneously clamped at their baseline activation level. No correlation between the synthesized patterns and specific human emotional, physiological, and/or brain states has been described or suggested in connection with these efforts.

Central nervous system (CNS) disorders are among the top causes of death and disability worldwide, and the disease burden has risen steeply over the past decade. It is predicted that over 50% of people in the U.S. will develop a CNS disorder over their lifetime. Within CNS, acute pain is one of the most common reasons for seeking medical care, resulting in over 115 million emergency department visits a year. Anxiety affects up to 40 million people, and many symptoms go unrecognized and/or untreated. Pain and anxiety are comorbid with multiple conditions, e.g., major depression, surgery, cancer, neuropathic pain. Fatigue-related conditions also affect many adults. For the millions of affected adults needing treatment, the combination of high cost, low efficacy, side effects, stigma and/or inconvenience of current drug therapies discourages their uptake and limits effectiveness in those treated.

CNS drug development is slow and expensive. The rate of new approvals is markedly lower than for other therapeutic areas. Progress is slowed by poor target validation, low specificity, absence of biomarkers, and difficulty in replicating trial results in real-world settings, especially in heterogeneous populations. Furthermore, CNS drug development is slow because existing platforms are generally unsuitable for targeting the brain's complex neural networks. Furthermore, a chemical compound developed in the context of a drug development program is not amenable to quick iteration and/or modification, which makes it difficult to optimize for a therapeutic effect.

Stimulation with light of various colors and frequencies has been shown to affect mood, diminish pain, and possibly reduce plaque formation in Alzheimer's patients. Images have been shown to be physiologically calming. However, neuroscientific research in the field of neural coding typically involves complex and expensive specialized equipment for the measurement of neuronal activity. Consequently, experimentation in this field is often done using a limited number of laboratory animals and/or human test subjects, which tends to limit the accuracy and general applicability of experimental results.

### SUMMARY

Disclosed embodiments provide a platform capable of generating safe, inexpensive therapeutic “dataceuticals” in the form of sensory stimuli, e.g., visual and/or audial, for therapeutic uses, such as for pain and anxiety relief. The platform uses artificial intelligence (AI) and real-time biofeedback to “read” (i.e., decipher) brain signals and “write” to (i.e., neuromodulate) the brain using dynamic visual neuromodulatory codes having specifically adapted patterns, colors, complexity, motion, and frequencies. Thus, approaches described herein, in effect, use AI-guided visual stimulation as a translational platform. To effectively and controllably “write” to the brain, visual information has to be parameterized.

Disclosed embodiments further provide a therapeutic-discovery platform capable of generating sensory stimuli, e.g., visual and/or audial stimuli, for a wide range of disorders. Dynamic visual neuromodulatory codes are viewed, e.g., on the screen of a laptop, smartphone, or VR headset, when a patient experiences symptoms. Designed to be inexpensive, noninvasive, and convenient to use, the sensory codes offer immediate and potentially sustained relief without requiring clinician interaction. Sensory codes are being developed for, inter alia, acute pain, fatigue and acute anxiety, thereby broadening potential treatment access for many who suffer pain or anxiety.

More generally, disclosed embodiments are directed to inducing specific states in the human brain to provide therapeutic benefits, as well as emotional and physiological benefits. For example, interactions between the brain and the immune system play an important role in neurological and neuropsychiatric disorders and many neurodegenerative and neurological diseases are rooted in dysfunction of the neuroimmune system. Therefore, manipulating this system has strong therapeutic potential. In disclosed embodiments, a stereotyped brain state is induced in a user to achieve a therapeutic result, such as, for example, affecting the heart rate of a user who has suffered a heart attack or causing neuronal distraction to help prevent onset of a seizure.

Disclosed embodiments may include techniques such as transfer and ensemble learning using artificial intelligence (AI), such as machine learning models and neural networks, e.g., convolutional neural networks, deep feedforward artificial neural networks, and adversarial neural networks, to develop better algorithms and produce generalizable therapeutic treatments. Instead of trying to create a perfect model of the brain, there is, in effect, a “chaining together” of a vast number of users to identify therapeutic treatments for large subsets of the users—such treatments being on par with pharmaceuticals in terms of their effectiveness in the general population. Accordingly, the therapeutic treatments developed in this manner can be delivered to patients without the need for individualized sensor measurements of, e.g., brain state and brain activity. This approach solves the problem of generalizability of treatment and results in reduced cost and other efficiencies in terms of the practical logistics of delivering therapeutic treatment.

The development of therapeutic treatments may be done in phases, which are summarized here and discussed in further detail below. The phases may occur in various orders and with repetition, e.g., iterative repetition, of one or more of the phases.

In one phase, a target state is established, which may be a desirable state which the therapeutic treatment is adapted to achieve, such as, for example, reduced anxiety (resulting in a reduced heart rate) or a “negative target” which the therapeutic treatments are adapted to avert, such as, for example, a brain state associated with migraine or seizure. The target state may be a brain state but may also, or alternatively, involve other indices and/or measures, e.g., heart rate, blood pressure, etc., indicative of underlying physiological conditions, such as hypertension, tachycardia, etc. Another brain state of interest is that of anesthetization, in which the therapeutic treatment is adapted to apply an alternative to conventional anesthesia to lock out all pain. Notably, in rats, anesthetizing works not by shutting down the brain but by, in effect, changing its frequencies. This therapeutic approach impacts aspects of pain processing, as well. Sensor measurements and various types of diagnostic imaging done while patients are in a target state may form the basis of a data set used to identify generalizable therapeutic results.

In disclosed embodiments, the target brain state may be achieved and characterized by: (i) inducing the target state in a patient (e.g., a user or test participant) and making measurements; or (ii) “surveying,” e.g., monitoring, the state of a participant using sensor mapping (e.g., a constellation of brain activity and physiological sensors) until the target state occurs. Various types of measurements are performed while the participant is the target state, such as, for example, brain imaging and physiological sensor readings, to provide a reference for identifying the target state.

The inducing of the target state may be done in various ways, including using drugs or other forms of stimulation (e.g., visual stimulation). For example, the participant may be asked to run or perform some other aerobic activity to achieve an elevated heart rate and a corresponding “negative target” physiological state which treatment will seek to move away from. As a further example, a participant may be presented with funny videos and/or images to induce a happy and/or low anxiety brain state. Taking migraines as an example, to facilitate more rapid experimentation, it would be helpful to be able to induce the condition, i.e., the negative target state, in a healthy subject. This could involve inducing pain to simulate a migraine condition. Various other conditions also have “comparable states” which can be used in the experimental setting to establish target states.

Isolating a target state using surveying, e.g., using sensor mapping, may include determining the difference in measured characteristics between a healthy person, e.g., a person not having a migraine or not experiencing depression, and a patient experiencing a corresponding target state. Furthermore, just as a target state can be induced in multiple ways, it is also possible to survey states through various methods, including disease diagnosis. The surveying may include establishing a patient type and state through sensor mapping. This is important in optimizing treatment, because a patient may have a specific disease, illness, or problem, but will also be at a particular on a curve of severity and may be moving up or down that curve. The sensor mapping of patient type and state is also important in considering response to treatment over time, such as a decrease in response over time. For example, depending on the stimuli or the treatment a patient has received, it may be found that the patient does not respond well—or at all—to the treatment. Therefore, consideration of “responders” and “non-responders” and the profiling of the patient and/or the disease is important.

Considering clinical trials as an analogy, the results of clinical trials comparing a new treatment with a control are based on an overall summary measure over the whole population enrolled that is assumed to apply to each treated patient, but this treatment effect can vary according to specific characteristics of the patients enrolled. The aim of “personalized medicine” is the tailoring of medical treatment to the individual characteristics of each patient in order to optimize individuals' outcomes. The key issue for personalized medicine is finding the criteria for an early identification of patients who can be responders and non-responders to each therapy. In contrast to this, the disclosed embodiments are directed to analyzing individual outcomes to determine a generalizable effect, such that a particular treatment is likely to be effective for a large number of potential patients. To make such a determination, it is useful to classify individual participants as responders and non-responders, as noted above, and use these classifications to determine a summary measure for a population based on the individual treatment results, especially results involving a high ratio of responders to non-responders.

In another phase, a patient (i.e., a user) is presented with visual neuromodulatory codes while in a state other than the target state—which may be deemed a “current state”—to induce a specific target state. This phase may be considered to be a therapeutic treatment phase, because the user receives the therapeutic benefits of the target state. Alternatively, in a case in which the target state is an undesirable state, e.g., migraine, the visual neuromodulatory codes are presented with the objective of moving the patient away from the target state.

In another phase, temporal and contextual reinforcement are performed while the user is receiving treatment. The reinforcement encompasses feedback of measured brain state and physiological conditions of the user and, based on this feedback, the therapeutic treatment may be adjusted to increase its effectiveness. In some cases, a particular treatment may not be entirely effective for a particular user. For example, a patient experiencing depression may require more than therapy adapted to increase happiness, because the patient's condition may have a number of different bases. The effectiveness of the therapy is based at least in part on a comparison of the various measured characteristics of the patient over time and in changing contexts (i.e., environments) compared to a reference healthy patient. This allows for the treatment to be reinforced (i.e., refined or optimized) over time as more temporal and contextual data becomes available to account for external influences which may affect the effectiveness of a treatment regime. This, in effect, establishes a learning (or “reinforcement”) phase. A response curve may be created to allow this technique to be applied beyond the range of what has been directly measured.

In the case of treatment of epileptic seizures, which can be difficult to predict, it may be possible to predict such seizures at least a few minutes in advance given sufficient temporal and environmental data. This would allow treatment, e.g., in the form of a specific visual stimulus, to avert and/or lessen the severity of a seizure. Furthermore, the treatment could be adjusted to achieve increased effectiveness by, for example, adjusting the advance warning time so the treatment can be delivered at an optimal time relative to the predicted onset. As a further example, temporal and contextual data may indicate that a user's anxiety levels increase when the user views specific types of content, e.g., particular types of videos. The system learns to associate these types of content with specific visual neuromodulatory codes which can be overlaid on—without obscuring—content as it is delivered to the user. Visual neuromodulatory codes could have various predefined strengths and/or doses and could be dynamic to adapt to changing circumstances of the patient's states.

Another phase uses transfer learning to allow the accumulated knowledge of the treatment artificial intelligence to be applied to new target states, e.g., target brain states, and new therapeutic applications. “Transfer learning” involves generalizing or transferring generalized knowledge gained in one context to novel, previously unseen domains. For example, a progressive network can transfer knowledge gained in one context, e.g., treatment of a particular patient and/or condition, to learn rapidly (i.e., reduce training time) in treatment of another patient and/or condition. The use of transfer learning, with system-level labeling of stimuli, provides a substantial advantage in terms of the specificity of the system. For example, for a treatment regime involving a specific kind of neuronal population or brain state which has not been handled previously, a selection of visual neuromodulatory codes can be made within a reduced problem space, as opposed to selecting from an entire “stimuli library.” Furthermore, the use of transfer learning leverages existing data collected from other patients to build a model for new patients with little calibration data. In some cases, a conditional transfer learning framework may be used to facilitate a transfer of labeled data from one patient to another, thereby improving subject-specific performance. The conditional framework assesses a patient's transferability for positive transfer (i.e., a transfer which improves subject-specific performance without increasing the labeled data) and then selectively leverages the data from patients with comparable feature spaces.

Disclosed embodiments involve the use of non-figurative (i.e., abstract, non-semantic, and/or non-representational) visual stimuli, such as the visual neuromodulatory codes described herein, which have advantages over figurative content. Non-figurative visual stimuli can be brought under tight experimental control for the purpose of stimulus optimization. Under AI guidance, specific features (e.g., shape, color, duration, movement, frequency, hue, etc.) can be expressed as parameters and gradually readjusted and recombined, frame by frame, pixel by pixel, to drive bioresponse in the desired direction. Unlike pictures of people or scenes, non-figurative visual stimuli are free of cultural or language bias and thus more generalizable as a global therapeutic.

In disclosed embodiments, there are various methods of delivery for the visual neuromodulatory codes, including presenting on a display but running in the background, “focused delivery” (e.g., user focuses on stimulus for a determined time with full attention), and overlaid—additive (e.g., a largely translucent layer overlaid on video or web browser content). The method of delivery may be determined based on temporal and contextual reinforcement considerations, in which case the delivery method is depends on how best to reinforce and optimize the treatment. For example, a user may be watching video content that is upsetting, but the system has learned to deliver visual neuromodulatory codes by overlaying it on the video content to neutralize any negative sentiment, response, or symptoms. For example, an overlay on content may make a screen look noisier but a user generally would not notice non-semantic content presented in this manner. As a further example, visual neuromodulatory codes could be overlaid on text presented on a screen without occupying the white space between letters and, thus, would not interfere with reading. In disclosed embodiments, the method of delivery may involve a user being presented with an augmented reality session while walking around. In such a case, when the user comes upon a landmark, e.g., a friend's house, which triggers a negative state, e.g., addictive behavior, the system may overlay visual neuromodulatory codes which induce positive feelings and/or distracts the user to look elsewhere.

To activate specific targeted areas in the visual cortex, neuronal selectivity can be examined using the vast hypothesis space of a generative deep neural network, without assumptions about features or semantic categories. A genetic algorithm can be used to search this space for stimuli that maximize neuronal firing and/or feedback data indicative of responses of a user, or group of participants, during display of the stimuli. This allows for the evolution of synthetic images of objects with complex combinations of shapes, colors, and textures, sometimes resembling animals or familiar people, other times revealing novel patterns that do not map to any clear semantic category.

In disclosed embodiments, a combination of a pre-trained deep generative neural network and a genetic algorithm can be used to allow neuronal responses and/or feedback data indicative of responses of a user, or group of participants, during display of the stimuli to guide the evolution of synthetic images. By training large numbers of images, a generative adversarial network can learn to model the statistics of natural images without merely memorizing the training set, thus representing a vast and general image space constrained only by natural image statistics. This provides an efficient space in which to perform a genetic algorithm, because the brain also learns from real-world images, so its preferred images are also likely to follow natural image statistics.

Convolutional neural networks have been shown to emulate aspects of computation along the primate ventral visual stream. Particular generative networks have been used to synthesize images that strongly activate units in various convolutional neural networks. In disclosed embodiments, an adversarial generative network may be used, having an architecture of a pre-trained deep generative network with, for example, a number of fully connected layers and a set of deconvolutional modules. The generative network takes vectors, e.g., 4,096-dimensional vectors (image codes) as input and deterministically transforms them into images, e.g., 256×256 RGB images. In conjunction with this, a genetic algorithm can use responses of neurons recorded and/or feedback data indicative of responses of a user, or group of participants, during display of the images to optimize image codes input to this network.

In disclosed embodiments, therapeutic visual neuromodulatory codes may be delivered by streaming dynamic codes to the user. Among the advantages of presenting the stimuli as dynamic video or visual information is that it helps prevent desensitization of the user to the stimuli, e.g., by presenting combinations of different types of visual neuromodulatory codes. The use of streaming to deliver the therapeutic treatment allows connection, i.e., personalization of the streaming content to a particular user to prevent abuse, e.g., overuse or “overdose” of the treatment. For example, one particular user's face can be linked to the delivery of the streaming service, thereby preventing the abuse of the system. Streaming services can also support dynamic, embedded watermarking to prevent copyright theft. Streaming services can also be adapted to deliver visual neuromodulatory codes, with or without accompanying content, at high frame rates to help prevent video recording. In disclosed embodiments, the streaming content may be downloaded onto a user's device, e.g., a mobile phone. There can be processing at the server side, the user-device side, or both. The specific nature of the processing can be informed by the sensor mapping and the patient type and state information, including processing relating to temporal and contextual reinforcement, as discussed above. If the user has an Internet connection, the data feeds (i.e., the visual neuromodulatory codes and other content) can be provided by a remote server to the user's mobile device. Alternatively, the data feeds could be generated on the user's mobile device in the absence of an Internet connection.

A broad aspect of the present disclosure is a method to generate non-figurative visual neuromodulatory codes adapted to produce physiological responses having therapeutic or performance-enhancing effects. The method includes rendering a visual neuromodulatory code based on a set of rendering parameters. The method further includes outputting the visual neuromodulatory code to be displayed on a plurality of electronic screens to be viewed simultaneously by a plurality of subjects. The method further includes receiving output of one or more sensors that measure, during the outputting of the visual neuromodulatory code, one or more physiological responses of each of the plurality of subjects. The method further includes calculating a value of an outcome function based on the one or more physiological responses of each of the plurality of subjects. The method further includes determining an updated predictive model based at least in part on a current predictive model and the calculated value of the outcome function—the predictive model providing an estimated value of the outcome function for a given set of rendering parameters. The method further includes calculating values for a set of adapted rendering parameters. The method is iteratively repeated using the set of adapted rendering parameters, until a defined set of stopping criteria are satisfied, to produce an adapted visual neuromodulatory code. The method further includes outputting, upon satisfying the defined set of stopping criteria, the adapted visual neuromodulatory code based on the set of adapted rendering parameters.

In some embodiments, the outcome function is indicative of: a therapeutic effectiveness of the visual neuromodulatory code.

In some embodiments, the outcome function is indicative of a degree of generalizability, among the plurality of subjects, of the therapeutic effectiveness of the visual neuromodulatory code.

In some embodiments, the rendering the visual neuromodulatory code based on the set of rendering parameters comprises projecting a latent representation of the visual neuromodulatory code onto a parameter space of a rendering engine.

In some embodiments, the calculating of values for a set of adapted rendering parameters based at least in part on: determining, using the updated predictive model, an estimated value of the outcome function for a plurality of values of the set of rendering parameters to form a response characteristic; and determining values of the set of adapted rendering parameters based at least in part on the response characteristic.

In some embodiments, the determining of values of the set of adapted rendering parameters comprises applying an acquisition function to the response characteristic to optimize selection of the values of the set of adapted rendering parameters.

In some embodiments, the method includes characterizing a sample visual neuromodulatory code using a plurality of defined descriptive spaces, each including one or more descriptive parameters. The characterizing comprises analyzing the sample visual neuromodulatory code to determine values of the descriptive parameters of each of the plurality of defined descriptive spaces. The performance of each of the plurality of defined descriptive spaces is modeled. One of the plurality of defined descriptive spaces is selected based at least in part on the modeling to define constituent parameters of the set of rendering parameters.

In some embodiments, the modeling of the performance of each of the plurality of defined descriptive spaces comprises using a Bayesian optimization algorithm.

In some embodiments, a first descriptive space, of the plurality of defined descriptive spaces, comprises low-level statistics of the sample visual neuromodulatory code, including at least one of color, brightness, and contrast.

In some embodiments, a second descriptive space, of the plurality of defined descriptive spaces, comprises metrics characterizing visual content of the sample visual neuromodulatory code, including at least one of spatial frequencies and scene complexity.

In some embodiments, a third descriptive space, of the plurality of defined descriptive spaces, comprises intermediate representations of visual content of the sample visual neuromodulatory code, the intermediate representations produced by processing the sample visual neuromodulatory code using a convolutional neural network trained to perform object recognition and encoding of visual information.

In some embodiments, in the receiving output of the one or more sensors, the one or more sensors are adapted to measure at least one of the following: neurological responses, physiological responses, and behavioral responses.

In some embodiments, the one or more sensors include one or more of the following: electroencephalogram (EEG), quantitative EEG, magneto-encephalography (MEG), single-photon emission computed tomography (SPECT), positron emission tomography (PET), functional magnetic resonance imaging (fMRI), functional near-infrared spectroscopy (fNIRS), EMG, electrocardiogram (ECG), pulse rate, blood pressure, and galvanic skin response (GSR).

In some embodiments, the method is repeated to produce a plurality of adapted visual neuromodulatory codes and further includes forming a dynamic adapted visual neuromodulatory code based at least in part on the plurality of adapted visual neuromodulatory codes.

In some embodiments, the forming of a dynamic adapted visual neuromodulatory code includes combining the plurality of adapted visual neuromodulatory codes to form a sequence of adapted visual neuromodulatory codes.

In some embodiments, the forming of a dynamic adapted visual neuromodulatory code further includes processing the plurality of adapted visual neuromodulatory codes to form intermediate images in the sequence of adapted visual neuromodulatory codes.

In some embodiments, the stopping criteria are based on at least one of: a predefined number of iterations, characteristics of the acquisition function, and a determination that convergence of the outcome function with target criteria will not occur within a defined number of iterations.

In some embodiments, a system generates non-figurative visual neuromodulatory codes adapted to produce physiological responses having therapeutic or performance-enhancing effects. The system includes at least one processor and at least one non-transitory processor-readable medium that stores processor-executable instructions which, when executed by the at least one processor, cause the at least one processor to perform the method of the broad aspect discussed above.

In some embodiments, a method provides non-figurative visual neuromodulatory codes adapted to produce physiological responses having therapeutic or performance-enhancing effects. This method includes retrieving one or more adapted visual neuromodulatory codes, the one or more adapted visual neuromodulatory codes being adapted to produce physiological responses having therapeutic or performance-enhancing effects; and outputting to an electronic display of a device viewable by a user the one or more adapted visual neuromodulatory codes, wherein the one or more adapted visual neuromodulatory codes are generated by performing the method of the broad aspect discussed above.

In some embodiments, the retrieving of the one or more adapted visual neuromodulatory codes includes receiving the one or more adapted visual neuromodulatory codes via a network or retrieving the one or more adapted visual neuromodulatory codes from a memory of the user device.

In some embodiments, in the outputting to the electronic display of the user device the one or more adapted visual neuromodulatory codes, each of the one or more adapted visual neuromodulatory codes is displayed for a determined time period, the determined time period being adapted based on user feedback data indicative of responses of the user.

In some embodiments, the outputting to the electronic display of the user device the one or more adapted visual neuromodulatory codes includes combining the one or more adapted visual neuromodulatory codes with displayed content.

In some embodiments, the displayed content includes at least one of: displayed output of an app, displayed output of a browser, and a user interface of the user device.

In some embodiments, this method further includes obtaining user feedback data indicative of responses of the user during the outputting to an electronic display of the user device the one or more adapted visual neuromodulatory codes.

In some embodiments, the obtaining user feedback data indicative of responses of the user includes using components of the user device to perform at least one of: measuring voice stress levels, detecting movement, tracking eye movement, and receiving input to displayed prompts.

In some embodiments, the obtaining user feedback data indicative of responses of the user includes receiving data from a wearable neurological sensor.

Another broad aspect of the present disclosure is a method to generate visual neuromodulatory codes to produce physiological responses having therapeutic or performance-enhancing effects. The method includes presenting a first set of visual stimulus images, such as visual neuromodulatory codes, to a subject while measuring physiological responses of the subject and classifying the first set of visual stimulus images into classes based on the measured physiological responses of the subject. The method further includes generating, for at least one specified class of the classes, a latent space representation of visual stimulus images in the at least one specified class. The method further includes generating a second set of visual stimulus images based at least in part on the latent space representation of the visual stimulus images in the at least one specified class and incorporating the second set of visual stimulus images into a third set of visual stimulus images. The method further includes iteratively repeating, using the third set of visual stimulus images, the classifying the visual stimulus images, the generating the latent space representation, the generating the second set of visual stimulus images, and the combining until a change in the latent space representation of the visual stimulus images in the at least one specified class, from one iteration to a next iteration, is within a defined range. The method further includes outputting the third set of visual stimulus images as visual neuromodulatory codes.

This aspect of the present disclosure may form the basis of an implementation in its own right, as described in the detailed description, or it may be used in combination with any of the embodiments disclosed herein. In some embodiments, this aspect of the present disclosure may be used in combination with the method to generate non-figurative visual neuromodulatory codes adapted to produce physiological responses having therapeutic or performance-enhancing effects. In some embodiments, this aspect of the present disclosure may be used in rendering a visual neuromodulatory code based on a set of rendering parameters.

In some embodiments, at least a portion of the first set of visual stimulus images is generated randomly.

In some embodiments, the classifying of the first set of visual stimulus images into classes based on the measured physiological responses of the subject comprises detecting irregularities in at least one of a time domain and a time-frequency domain of the measured physiological responses of the subject.

In some embodiments, the generating of the latent space representation is performed using a convolutional neural network.

In some embodiments, the generating of a second set of visual stimulus images comprises using a pre-trained neural network.

### BRIEF DESCRIPTION OF THE DRAWINGS

FIG. 1 depicts an embodiment of a system to generate and optimize non-figurative visual neuromodulatory codes implemented using an “inner loop” which optimizes visual neuromodulatory codes through biomedical sensor feedback to maximize the therapeutic impact for an individual subject or group of subjects and an “outer loop” which uses various processing techniques to generalize the effectiveness of the visual neuromodulatory codes produced by the inner loop for the general population of users.

FIG. 2 depicts an embodiment of a system to generate non-figurative visual neuromodulatory codes adapted to produce physiological responses having therapeutic or performance-enhancing effects.

FIG. 3 depicts an embodiment of a method, usable with the system of FIG. 2, to generate visual neuromodulatory codes to produce physiological responses having therapeutic or performance-enhancing effects.

FIG. 4 depicts an embodiment of a method, usable with the system of FIG. 18, to provide visual neuromodulatory codes to produce physiological responses having therapeutic or performance-enhancing effects.

FIG. 5 depicts an embodiment of a system to generate and provide to a user a visual stimulus, using visual codes displayed to a group of participants, to produce physiological responses having therapeutic or performance-enhancing effects.

FIG. 6 depicts an embodiment of a method, usable with the system of FIG. 5, to generate a visual stimulus to produce physiological responses having therapeutic or performance-enhancing effects.

FIG. 7 depicts an initial population of images created from random achromatic textures constructed from a set of textures which are derived from randomly sampled photographs of natural objects on a gray background.

FIG. 8 depicts an embodiment of a system to generate a visual stimulus, using brain state data and/or brain activity data measured while visual codes are displayed to a participant in a target state and a current state, to produce physiological responses having therapeutic or performance-enhancing effects.

FIG. 9 depicts an embodiment of a method, usable with the system of FIG. 8, to generate a visual stimulus to produce physiological responses having therapeutic or performance-enhancing effects.

FIG. 10 depicts an embodiment of a system to deliver a visual stimulus, generated using visual codes displayed to a group of participants, to produce physiological responses having therapeutic or performance-enhancing effects.

FIG. 11 depicts formation of a visual stimulus by overlaying a visual code on content displayable on an electronic device, as in the system of FIG. 10.

FIG. 12 depicts an embodiment of a method to deliver a visual stimulus, usable with the system of FIG. 10, to produce physiological responses having therapeutic or performance-enhancing effects.

FIG. 13 depicts an embodiment of a system to deliver a visual stimulus, generated using brain state data and/or brain activity data measured while visual codes are displayed to a participant in a target state and a current state, to produce physiological responses having therapeutic or performance-enhancing effects.

FIG. 14 depicts an embodiment of a method to deliver a visual stimulus, usable with the system of FIG. 13, to produce physiological responses having therapeutic or performance-enhancing effects.

FIG. 15 depicts an embodiment of a system to generate visual neuromodulatory codes with closed-loop approach using an optimized descriptive space.

FIG. 16 depicts an embodiment of a method, usable with the system of FIG. 15, to generate visual neuromodulatory codes with closed-loop approach using an optimized descriptive space.

FIG. 17 depicts an embodiment of a method to determine an optimized descriptive space to characterize visual neuromodulatory codes.

FIG. 18 depicts an embodiment of a system to deliver visual neuromodulatory codes generated with closed-loop approach using an optimized descriptive space.

FIG. 19 depicts an embodiment of a method, usable with the system of FIG. 18, to deliver visual neuromodulatory codes generated with closed-loop approach using an optimized descriptive space according to the method of FIG. 16.

FIG. 20 depicts an embodiment of a system to generate visual neuromodulatory codes by reverse correlation and stimuli classification.

FIG. 21 depicts an embodiment of a method, usable with the system of FIG. 20 to generate visual neuromodulatory codes by reverse correlation and stimuli classification.

FIG. 22 depicts an embodiment of a method, usable with the system of FIG. 18, to deliver visual neuromodulatory codes generated by reverse correlation and stimuli classification according to the method of FIG. 21.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with computer systems, server computers, and/or communications networks have not been shown or described in detail to avoid unnecessarily obscuring descriptions of the implementations.

Unless the context requires otherwise, throughout the specification and claims that follow, the word “comprising” is synonymous with “including,” and is inclusive or open-ended (i.e., does not exclude additional, unrecited elements or method acts). Reference throughout this specification to “one implementation” or “an implementation” or “particular implementations” means that a particular feature, structure or characteristic described in connection with the implementation is included in at least one implementation. Thus, the appearances of the phrases “in one implementation” or “in an implementation” or “particular implementations” in various places throughout this specification are not necessarily all referring to the same implementation. Furthermore, the particular features, structures, or characteristics may be combined in any suitable manner in one or more implementations.

As used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise. The headings and Abstract of the Disclosure provided herein are for convenience only and do not interpret the scope or meaning of the implementations.

Physiology is a branch of biology that deals with the functions and activities of life or of living matter (e.g., organs, tissues, or cells) and of the physical and chemical phenomena involved. It includes the various organic processes and phenomena of an organism and any of its parts and any particular bodily process. Hence, the term “physiological” is used herein to broadly mean characteristic of or appropriate to the functioning of an organism, including human physiology. The term includes the characteristics and functioning of the nervous system, the brain, and all other bodily functions and systems.

The term “neurophysiology” refers to the physiology of the nervous system. The term “neural” and the prefix “neuro” likewise refer to the nervous system. As used herein, all of these terms and prefixes refer to the physiology of the nervous system and brain. In some instances, these terms and prefixes are used herein to refer to physiology more generally, including the nervous system, the brain, and physiological systems which are physically and functionally related to the nervous system and the brain.

Embodiments discussed herein provide: (a) a therapeutic discovery platform; and (b) a library of therapeutic visual neuromodulatory codes (“dataceuticals”) produced by the platform. The therapeutic discovery platform, guided by artificial intelligence (AI), carries out search and discovery for therapeutic visual neuromodulatory codes, which are optimized and packaged as a low-cost, safe, rapidly acting, and effective visual neuromodulatory codes for prescription or over-the-counter use.

The therapeutic discovery platform is designed to support the discovery of effective therapeutic stimulation for various conditions. At the heart of its functionality is a loop wherein stimulation parameters are continuously adapted, based on physiologic response derived from biofeedback (e.g., closed-loop adaptive visual stimulation), to reach a targeted response. The platform comprises three major components: (1) a “generator” to produce a wide range of visual neuromodulatory codes with the full control of parameters such as global structure of an image, details and fine textures, and coloring; (2) a sensor subsystem for real-time measurement of physiologic feedback (e.g., heart, brain and muscle response); and (3) an analysis subsystem that analyzes the biofeedback and adapts the stimulation parameters, e.g., by adapting rendering parameters which control the visual neuromodulatory codes produced by the generator.

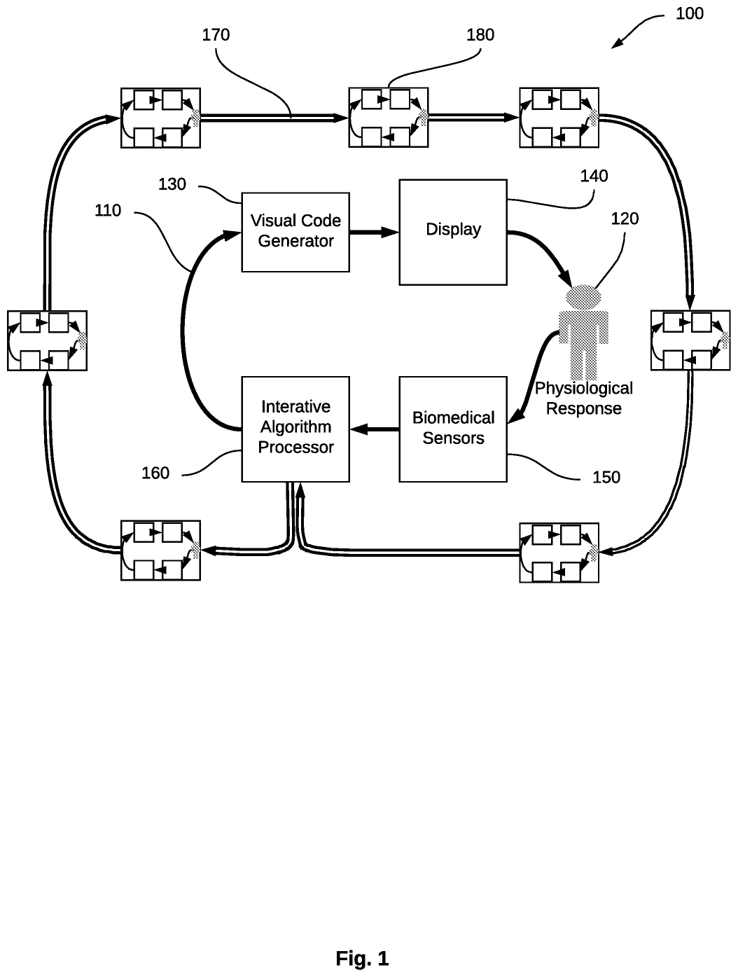

FIG. 1 depicts an embodiment of a system 100 to generate and optimize visual neuromodulatory codes to produce physiological responses having therapeutic or performance-enhancing effects. The system 100 combines visual synthesis technologies, real-time physiological feedback (including neurofeedback) processing, and artificial intelligence guidance to generate stimulation parameters to accelerate discovery and optimize therapeutic effect of visual neuromodulatory codes. The system is implemented in two stages: an “inner loop” which optimizes visual neuromodulatory codes through biomedical sensor feedback to maximize the therapeutic impact for an individual subject or group of subjects; and an “outer loop” which uses various processing techniques to generalize the effectiveness of the visual neuromodulatory codes produced by the inner loop for the general population of users. It should be noted that although the phrase “therapeutic or performance-enhancing effects” is used throughout the present application, in some cases an effect may have both a therapeutic and a performance-enhancing aspect, so it should be understood that physiological responses may have therapeutic or performance-enhancing effects or both. The term “performance-enhancing” refers to effects such as stimulation (i.e., as with caffeine), improved focus, improved attention, etc.

In embodiments, to maximize the chances of discovering responses that are consistent across subjects, optimization may be carried out on a group basis, in which case a group of subjects is presented simultaneously with visual images in the form of visual neuromodulatory codes. The bio-responses of the group of subjects are aggregated and analyzed in real time to determine which stimulation parameters (i.e., the parameters used to generate the visual neuromodulatory codes) are associated with the greatest response. The system optimizes the stimuli, readjusting and recombining the visual parameters to quickly drive the collective response of the group of subjects in the direction of greater response. Such group optimization increases the chances of evoking ranges of finely graded responses that have cross-subject consistency.

The system 100 includes an iterative inner loop 110 which synthesizes and refines visual neuromodulatory codes based on the physiological responses of an individual subject (e.g., 120) or group of subjects. The inner loop 110 can be implemented as specialized equipment, e.g., in a facility or laboratory setting, dedicated to generating therapeutic visual neuromodulatory codes. Alternatively, or in addition, the inner loop 110 can be implemented as a component of equipment used to deliver therapeutic visual neuromodulatory codes to users, in which case the subject 120 (or subjects) is also a user of the system.

The inner loop 110 includes a visual stimulus generator 130 to synthesize visual neuromodulatory codes, which may be in the form of a set of one or more visual neuromodulatory codes defined by a set of image parameters (e.g., “rendering parameters”). In implementations, the synthesis of the visual neuromodulatory codes may be based on artificial intelligence-based manipulation of image data and image parameters. The visual neuromodulatory codes are output by the visual stimulus generator 130 to a display 140 to be viewed by the subject 120 (or subjects). Physiological responses of the subject 120 (or subjects) are measured by biomedical sensors 150, e.g., electroencephalogram (EEG), pulse rate, and blood pressure, while the visual neuromodulatory codes are being presented to the subject 120 (or subjects).

The measured physiological data is received by an iterative algorithm processor 160, which determines whether the physiological responses of the subject 120 (or subjects) meet a set of target criteria. If the physiological responses of the subject 120 (or subjects) do not meet the target criteria, then a set of adapted image parameters is generated by the iterative algorithm processor 160 based on the output of the sensors 150. The adapted image parameters are used by the visual stimulus generator 130 to produce adapted visual neuromodulatory codes to be output to the display 140. The iterative inner loop process continues until the physiological responses of the subject 120 (or subjects) meet the target criteria, at which point the visual neuromodulatory codes have been optimized for the particular subject 120 (or subjects).

An “outer loop” 170 of the system 100 provides for the generalization of visual neuromodulatory codes from a wide-ranging population of subjects and/or users. In the generalization process, optimized image parameters from a number of instances of inner loops 180 are processed to produce a generalized set of image parameters which have a high likelihood of being effective for a large number of users. The generalized set of image parameters evolves over time as additional subjects and/or users are included in the outer loop 170. As more patients use the system 100, the outer loop uses techniques such as ensemble and transfer learning to distill visual neuromodulatory codes into “dataceuticals” and optimize their effects to be generalizable across patients and conditions. By encoding visual information in a manner similar to the visual cortex through the use of artificial intelligence, visual neuromodulatory codes can efficiently activate brain circuits and expedite the search for optimal stimulation, thereby creating, in effect, a visual language for interfacing with and healing the brain.

Among the advantages of the system 100 is that it effectively accelerates central nervous system (CNS) translational science, because it allows therapeutic hypotheses to be tested quickly and repeatedly through artificial intelligence—guided iterations, thereby significantly speeding up treatment discovery by potentially orders of magnitude and increasing the chances of providing relief to millions of untreated and undertreated people worldwide.

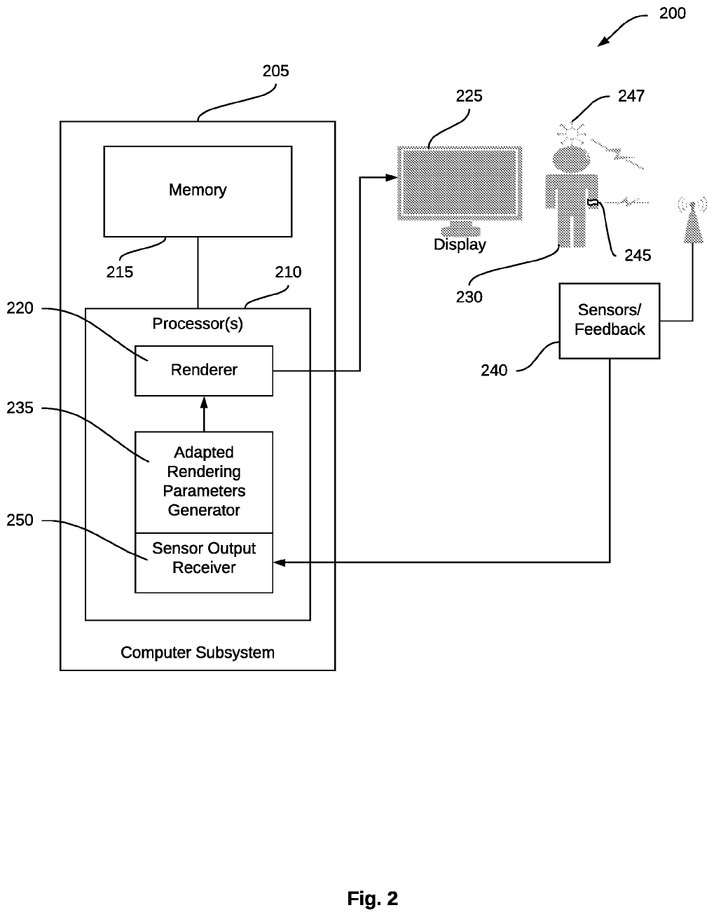

FIG. 2 depicts an embodiment of a system 200 to generate visual neuromodulatory codes to produce physiological responses having therapeutic or performance-enhancing effects (or both). The system 200 includes a computer subsystem 205 comprising at least one processor 210 and memory 215 (e.g., non-transitory processor-readable medium). The memory 215 stores processor-executable instructions which, when executed by the at least one processor 210, cause the at least one processor 210 to perform a method to generate the visual neuromodulatory codes. Specific aspects of the method performed by the processor 210 are depicted as elements (e.g., code, software modules, and/or processes) within the processor for purposes of discussion only.

The renderer 220 performs a rendering process to produce images (e.g., sequences of images) to be displayed on the display 225 by generating video data based on specific inputs. In implementations, the output of the rendering process is a digital image stored as an array of pixels. Each pixel value may be a single scalar component or a vector containing a separate scalar value for each color component. The renderer 220 may produce (i.e., synthesize) one or more visual neuromodulatory codes (e.g., a sequence of visual neuromodulatory codes) based on an initial set of rendering parameters (i.e., synthesis parameters) stored in the memory 215. The video data and/or signal resulting from the rendering is output by the computer subsystem 205 to the display 225.

The system 200 is configured to output the visual neuromodulatory codes to a display 225 viewable by a subject 230 or a number of subjects simultaneously. For example, a video monitor may be provided in a location where it can be accessed by the subject 230 (or subjects), e.g., a location where other components of the system are located. Alternatively, the video data may be transmitted via a network to be displayed on a video monitor or mobile device (not shown) of the subject (or subjects). In implementations, the subject 230 (or subjects) may be one of the users of the system.

In implementations, the system 200 may output to the display 225 a dynamic visual neuromodulatory code based on a plurality of visual neuromodulatory codes. For example, a dynamic visual neuromodulatory code may be formed by combining a number of visual neuromodulatory codes to form a sequence of visual neuromodulatory codes. In a further example, a dynamic visual neuromodulatory code may be adapted to produce at least one of the following effects: a pulsating effect, a zooming effect, a flickering effect, and a color-shift effect. In some cases, the formation of the dynamic visual neuromodulatory code may include processing a set, e.g., a sequence, of visual neuromodulatory codes to produce intermediate images in the sequence of visual neuromodulatory codes. Various techniques, such as interpolation of pixels and gaussian averaging, may be used to produce the intermediate images.

The system 200 includes one or more sensors 240, such as biomedical sensors, to measure physiological responses of the subject 230 (or subjects) while the visual neuromodulatory codes are being presented to the subject 230 (or subjects). For example, the system may include a wristband 245 and a head-worn apparatus 247 and may also include various other types of physiological and neurological feedback devices. In general, biomedical sensors include physical sensors, chemical sensors, and biological sensors. Physical sensors may be used to measure and monitor physiologic properties such as, for example, physical blood pressure, respiration, pulse, body temperature, heart sound, respiratory rate, blood viscosity, flow rate, flow rate, etc. Chemical sensors may be utilized to measure chemical parameters, such as, for example, oxygen and carbon dioxide concentration in the human metabolism, pH value, and ion levels in bodily fluids. Biological sensors (i.e., “biosensors”) are used to detect biological parameters, such as tissues, cells, enzymes, antigens, antibodies, receptors, hormones, cholic acid, acetylcholine, serotonin, DNA and RNA, and other proteins and biomarkers.

The sensors 240 used in the system 200 may include wearable devices, such as, for example, wristbands 245 and head-worn apparatuses 247. Other examples of wearable devices include smart glasses, watches, fitness bands/watches, running shoes, rings, armbands, belts, helmets, buttons, etc. In implementations, the physiological responses of the subject 230 (or subjects) may be measured using sensors adapted to measure, inter alia, one of the following: neurological responses, physiological responses, and behavioral responses. The sensors 240 may include one or more of the following: EEG, MEG, fMRI, ECG, EMG, electrocardiogram, pulse rate, and blood pressure. In some cases, wearable devices may identify a specific neural state, e.g., an epilepsy kindling event, thereby allowing the system to respond to counteract the state, artificial intelligence—guided visual neuromodulatory codes can be presented to counteract and neutralize the kindling with high specificity.

A sensor output receiver 250 of the computer subsystem 205 receives the outputs of the sensors 240, e.g., data and/or analog electrical signals, which are indicative of the physiological responses of the subject 230 (or subjects), as measured by the sensors 240 during the output of the visual neuromodulatory codes to the display 225. In implementations, the analog electrical signals may be converted into data by an external component, e.g., an analog-to-digital converter (ADC) (not shown). Alternatively, the computer subsystem 205 may have an internal component, e.g., an ADC card, installed to directly receive the analog electrical signals. Data output received from the sensors 240 in various forms and protocols, such as via a serial data bus or via network protocols, e.g., UDP or TCP/IP. The sensor output receiver 250 converts the sensor outputs, as necessary, into a form usable by the adapted rendering parameter generator 235.

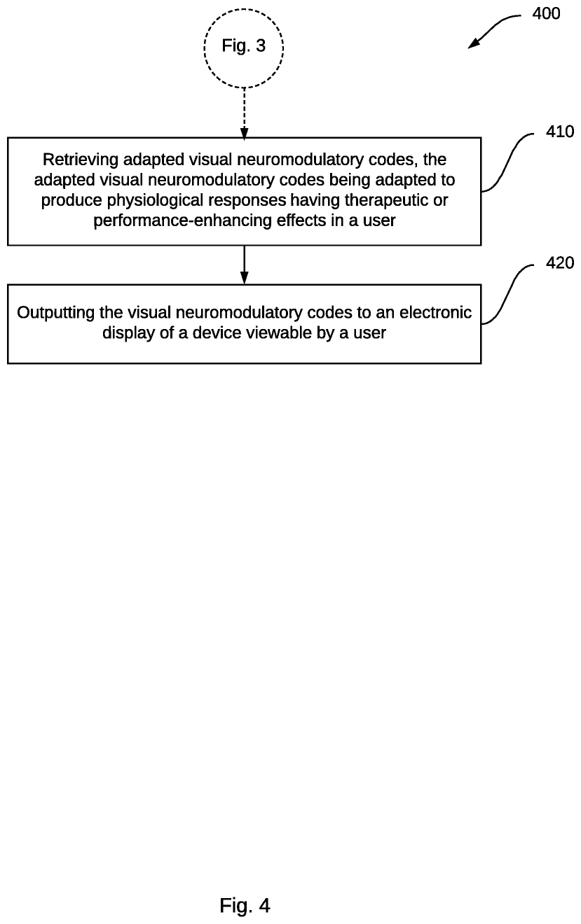

If measured physiological responses of the subject 230 (or subjects) do not meet a set of target criteria, the adapted rendering parameter generator 235 generates a set of adapted rendering parameters based at least in part on the received output of the sensors. The adapted rendering parameters are passed to the renderer 220 to be output to the display 225, as described above. The system 200 iteratively repeats the rendering (e.g., by the renderer 220), outputting the visual neuromodulatory codes to a display 225 viewable by the subject 230 (or subjects), and the receiving output of sensors 240 that measure, during the outputting of the visual neuromodulatory codes to the display 225, the physiological responses of the subject 230 using the adapted rendering parameters. The iterations are performed until the physiological responses of the subject 230 (or subjects), as measured by the sensors 240, meet the target criteria, at which point the system 200 outputs the visual neuromodulatory codes to be used in producing physiological responses having therapeutic or performance-enhancing effects (or both). In implementations, the adapted visual neuromodulatory codes may be used in a method to provide visual neuromodulatory codes (see, e.g., FIG. 4 and related description below).

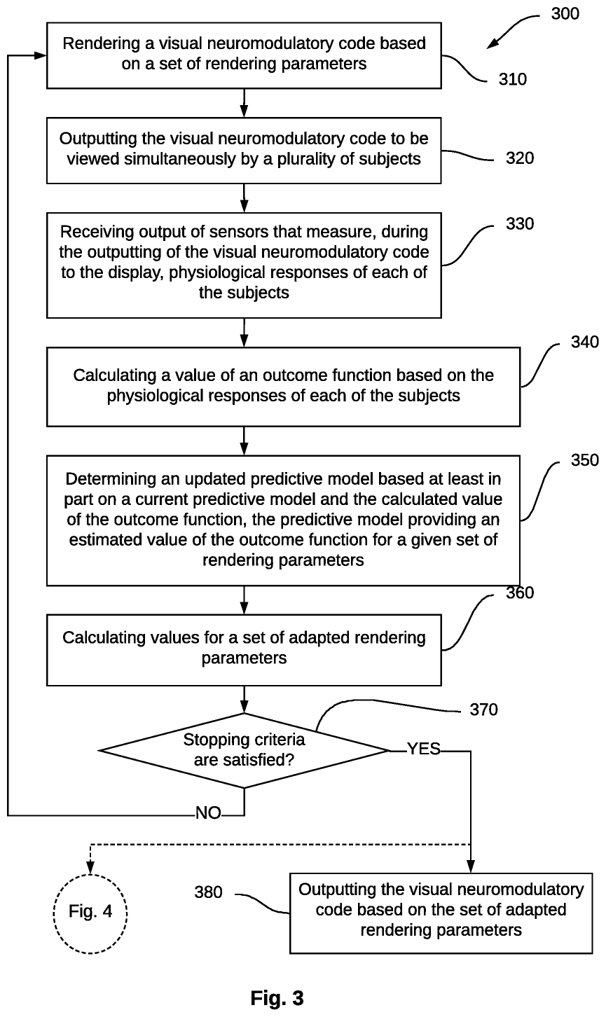

FIG. 3 depicts an embodiment of a method 300, usable with the system of FIG. 2, to generate visual neuromodulatory codes to produce physiological responses having therapeutic or performance-enhancing effects (or both).

In embodiments, a Bayesian optimization may be performed to adapt the rendering parameters—and hence optimize the resulting visual neuromodulatory codes—based on the physiological responses of the subjects. In particular, the optimization aims to drive the physiological responses of the subjects based on target criteria, which may be a combination of thresholds and/or ranges for various physiological measurements performed by sensors. For example, to achieve a therapeutic response which reduces stress, target criteria may be established which are indicative of a reduction in pulse rate and/or blood pressure. Using such an approach, the method can efficiently search through a large experiment space (e.g., the set of all possible rendering parameters) with the aim of identifying the experimental condition (e.g., a particular set of rendering parameters) that exhibits an optimal response in terms of physiological responses of subjects. In some embodiments, other analysis techniques, such as dynamic Bayesian networks, temporal event networks, and temporal nodes Bayesian networks, may be used to perform all or part of the adaptation of the rendering parameters.

The relationship between the experiment space and the physiological responses of the subjects may be quantified by an objective function (or “cost function”), which may be thought of as a “black box” function. The objective function may be relatively easy to specify but can be computationally challenging to calculate or result in a noisy calculation of cost over time. The form of the objective function is unknown and is often highly multi-dimensional depending on the number of input variables. For example, a set of rendering parameters used as input variables may include a multitude of parameters which characterize a rendered image, such as shape, color, duration, movement, frequency, hue, etc. In the example mentioned above, in which the goal is to achieve a therapeutic response which reduces stress, the objective function may be expressed in terms of neurophysiological features calculated from rate and/or blood pressure, e.g., heart rate variability and ratio systolic and diastolic blood pressure, each multiplied by scaling coefficients. In some embodiments, only a single physiological response may be taken into account by the objective function.

The optimization involves building a probabilistic model (referred to as the “surrogate function” or “predictive model”) of the objective function. The predictive model is progressively updated and refined in a closed loop by automatically selecting points to sample (e.g., selecting particular sets of rendering parameters) in the experiment space. An “acquisition function” is applied to the predictive model to optimally choose candidate samples (e.g., sets of rendering parameters) for evaluation with the objective function, i.e., evaluation by taking actual sensor measurements. Examples of acquisition functions include probability of improvement (PI), expected improvement (EI), and lower confidence bound (LCB).

The method 300 includes rendering a visual neuromodulatory code based on a set of rendering parameters (310). Various types of rendering engines may be used to produce the visual neuromodulatory code (i.e., image), such as, for example, procedural graphics, generative neural networks, gaming engines and virtual environments. Conventional rendering involves generating an image from a 2D or 3D model. Multiple models can be defined in a data file containing a number of “objects,” e.g., geometric shapes, in a defined language or data structure. A rendering data file may contain parameters and data structures defining geometry, viewpoint, texture, lighting, and shading information describing a virtual “scene.” While some aspects of rendering are more applicable to figurative images, i.e., scenes, the rendering parameters used to control these aspects may nevertheless be used in producing abstract, non-representational, and/or non-figurative images. Therefore, as used herein, the term “rendering parameter” is meant to include all parameters and data used in the rendering process, such that a rendered image (i.e., the image which serves as the visual neuromodulatory code) is completely specified by its corresponding rendering parameters.

In some embodiments, the rendering of the visual neuromodulatory code based on the set of rendering parameters may include projecting a latent representation of the visual neuromodulatory code onto the parameter space of a rendering engine. Depending on the rendering engine, the final appearance of the visual neuromodulatory code may vary, however the desired therapeutic properties are preserved.

The method further includes outputting the visual neuromodulatory code to be viewed simultaneously by a plurality of subjects (320). The method 300 further includes receiving output of one or more sensors that measure, during the outputting of the visual neuromodulatory code, one or more physiological responses of each of the plurality of subjects (330).

The method 300 further includes calculating a value of an outcome function based on the physiological responses of each of the plurality of subjects (340). The outcome function may act as a cost function (or loss function) to “score” the sensor measurements relative to target criteria. the outcome function is indicative of a therapeutic effectiveness of the visual neuromodulatory code.

The method 300 further includes determining an updated predictive model based at least in part on a current predictive model and the calculated value of the outcome function—the predictive model providing estimated value of the outcome function for a given set of rendering parameters (350).

The method 300 further includes calculating values for a set of adapted rendering parameters (360). The values may be calculated based at least in part on determining, using the updated predictive model, an estimated value of the outcome function for a plurality of values of the set of rendering parameters to form a response characteristic (e.g., response surface); and determining values of the set of adapted rendering parameters based at least in part on the response characteristic. In some embodiments, an acquisition function may be applied to the response characteristic to optimize selection of the values of the set of adapted rendering parameters.

The method 300 is iteratively repeated using the adapted rendering parameters until a defined set of stopping criteria are satisfied (370). Upon satisfying the defined set of stopping criteria, the visual neuromodulatory code based on the adapted rendering parameters is output (380). In implementations, the adapted visual neuromodulatory codes may be used in a method to deliver visual neuromodulatory codes (see, e.g., FIG. 4 and related description below).

As explained above, the outcome function (i.e., objective function) may be expressed in terms of neurophysiological features calculated from rate and/or blood pressure, e.g., heart rate variability and ratio systolic and diastolic blood pressure, each multiplied by scaling coefficients to produce a “score” to evaluate the rendering parameters in terms of target criteria, e.g., by determining a difference between the outcome function and a target value, threshold, and/or characteristic that is indicative of a desirable state or condition. Thus, the outcome function can be indicative of a therapeutic effectiveness of the visual neuromodulatory code.

As further discussed above (see, e.g., the discussion of FIG. 1), the system 100 provides for the generalization of visual neuromodulatory codes from a wide-ranging population of subjects and/or users. In the generalization process, optimized image parameters are processed to produce a generalized set of image parameters which have a high likelihood of being effective for a large number of users. In some embodiments, the outcome function may be indicative of a degree of generalizability, among the plurality of subjects, of the therapeutic effectiveness of the visual neuromodulatory code. For example, the outcome function may be defined to have a parameter relating to the variance of measure sensor data. This would allow the method to optimize for both therapeutic effect and generalizability.

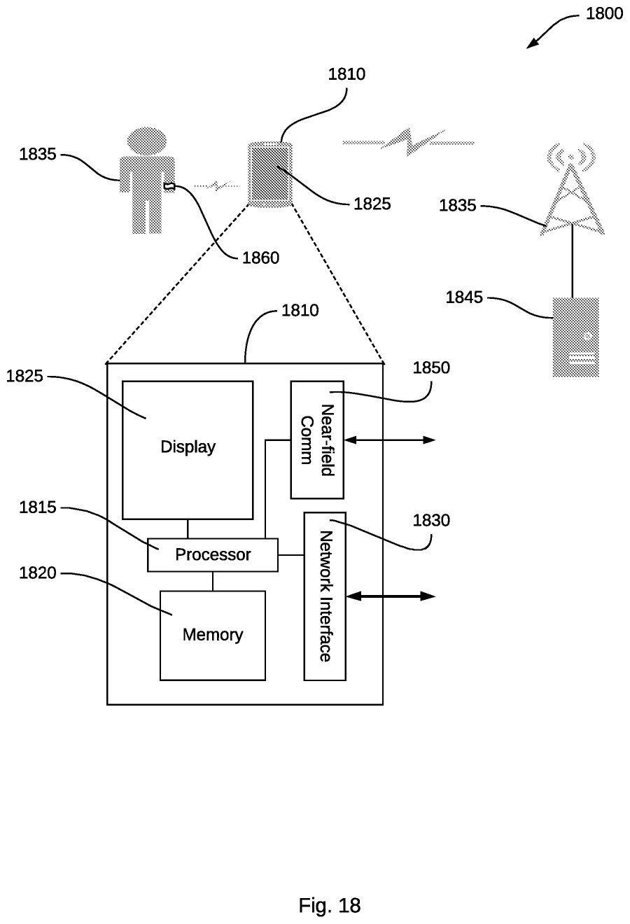

FIG. 4 depicts an embodiment of a method 400, usable with the system of FIG. 18, to provide visual neuromodulatory codes. The method 400 includes retrieving adapted visual neuromodulatory codes, which are adapted to produce physiological responses having therapeutic or performance-enhancing effects (410). The method 400 further includes outputting to an electronic display of a user device the adapted visual neuromodulatory codes (420). In implementations, the one or more adapted visual neuromodulatory codes may be generated, for example, according to the method of FIG. 3, discussed above.

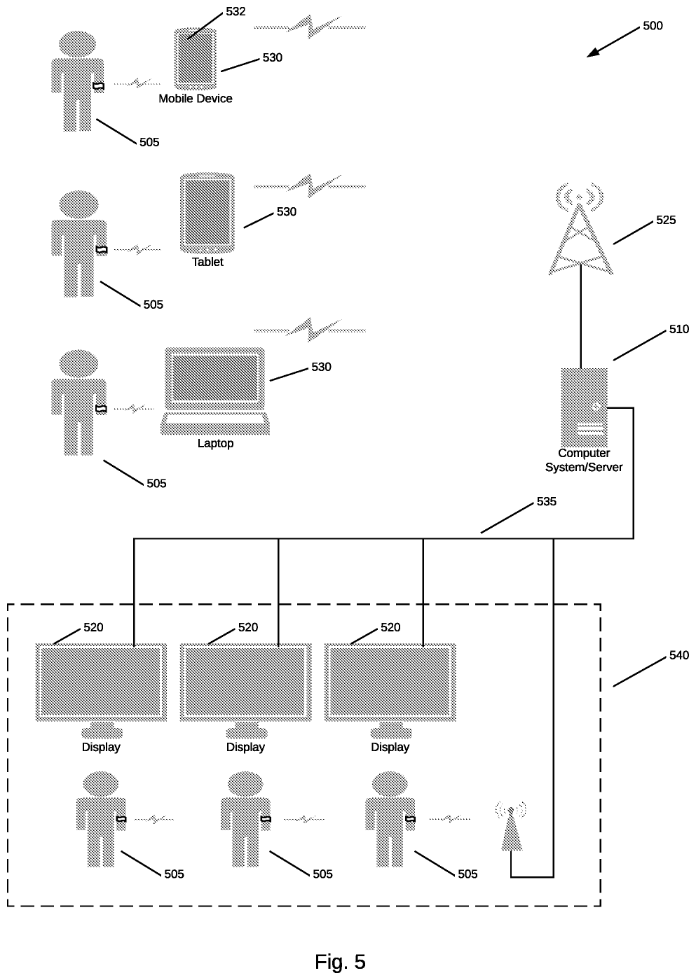

FIG. 5 depicts an embodiment of a system 500 to generate a visual stimulus, using visual codes displayed to a group of participants 505, to produce physiological responses having therapeutic or performance-enhancing effects. The system 500 is processor-based and may include a network-connected computer system/server 510 (and/or other types of computer systems) having at least one processor and memory/storage (e.g., non-transitory processor-readable medium such as random-access memory, read-only memory, and flash memory, as well as magnetic disk and other forms of electronic data storage). The memory/storage stores processor-executable instructions and data which, when executed by the at least one processor, cause the at least one processor to perform the necessary functions for the system to generate and provide to a user the visual stimulus.

A visual code or codes may be generated based on feedback from one or more participants 505 and used as the visual stimulus to produce physiological responses having therapeutic or performance-enhancing effects. The visual stimulus, or stimuli, generated in this manner may, inter alia, effect beneficial changes in specific human emotional, physiological, interoceptive, and/or behavioral states. The visual codes may be implemented in various forms and developed using various techniques, as described in further detail below. In alternative embodiments, other forms of stimuli may be used in conjunction with, or in lieu of, visual neuromodulatory codes, such as audio, sensory, chemical, and physical forms of stimulus

The visual code or codes are displayed to a group of participants 505—either individually or as a group—using electronic displays 520. For example, the server 510 may be connected via a network 525 to a number of personal electronic devices 530, such as mobile phones, tablets, and/or other types of computer systems and devices. The participants 505 may individually view the visual codes on an electronic display 532 of a personal electronic device 530, such as a mobile phone, simultaneously or at different times, i.e., the viewing by one user need not be done at the same time as other users in the group. The personal electronic device may be a wearable device, such as a fitness watch with a display or a pair of glasses that display images, e.g., virtual reality glasses, or other types of augmented-reality interfaces. In some cases, the visual code may be incorporated in content generated by an application running on the personal electronic device 530, such as a web browser. In such a case, the visual code may be overlaid on content displayed by the web browser, e.g., a webpage, so as to be unnoticed by a typical user.

Alternatively, the participants 505 may participate as a group in viewing the visual codes in a group setting on a single display or individual displays for each participant. In such a case, the server may be connected via a network 535 (or 525) to one or more electronic displays which allow for viewing of visual neuromodulatory codes by users in one or more facilities 540 set up for individual and/or group testing.

In some cases, the visual codes may be based at least in part on representational images. In other cases, the visual codes may be formed in a manner that avoids representational imagery. Indeed, the visual codes may incorporate content which is adapted to be perceived subliminally, as opposed to consciously. A “candidate” visual code may be used as an initial or intermediate iteration of the visual code. The candidate visual code, as described in further detail below, may be similar or identical in form and function to the visual code but may be generated by a different system and/or method.

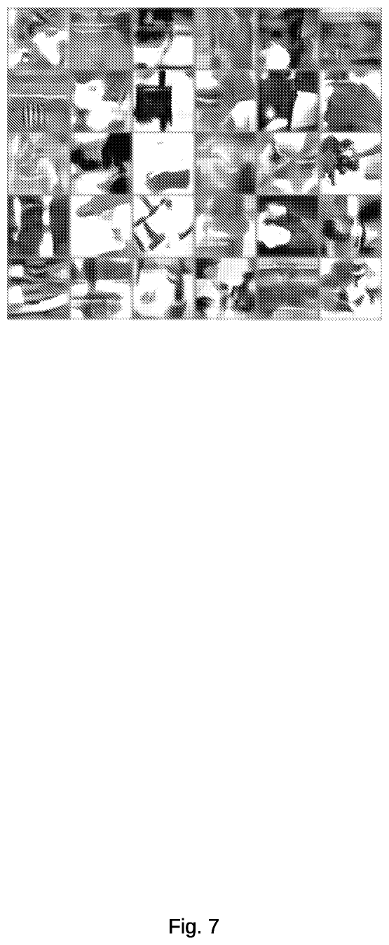

As shown in FIG. 7, the generation of images may start from an initial population of images (e.g., 40 images) created from random achromatic textures constructed from a set of textures which are derived from randomly sampled photographs of natural objects on a gray background. An initial set of “all-zero codes” can be optimized for pixel-wise loss between the synthesized images and the target images using backpropagation through a generative network for a number of iterations, with a linearly decreasing learning rate. The resulting image codes produced are, to an extent, blurred versions of the target images, due to the pixel-wise loss function, thereby producing a set of initial images having quasi-random textures.

Neuronal responses to each synthetic image and/or physiological feedback data indicative of responses of a user, or group of participants, during display of each synthetic image, are used to score the image codes. In each generation, images may be generated from the top (e.g., top 10) image codes from the previous generation, unchanged, plus new image codes (e.g., 30 new image codes) generated by mutation and recombination of all the codes from the preceding generation selected, for example, on the basis of feedback data indicative of responses of a user, or group of participants, during display of the image codes. In disclosed embodiments, images may also be evaluated using an artificial neural network as a model of biological neurons.

In some implementations, the visual codes may be incorporated in a video displayed to the users. In such a case, the visual codes may appear in the video for a sufficiently short duration so that the visual codes are not consciously noticed by the user or users. In various implementations, one or more of the visual codes may encompass all pixels of an image “frame,” i.e., individual image of the set of images of which the video is composed, such that the video is blanked for a sufficiently short duration so that the user does not notice that the video has been blanked. In some cases, the visual code or codes cannot be consciously identified by the user while viewing the video. Pixels forming a visual code may be arranged in groups that are not discernible from pixels of a remainder of an image in the video. For example, pixels of a visual code may be arranged in groups that are sufficiently small so that the visual code cannot be consciously noticed when viewed by a typical user.

The displayed visual code or codes are adapted to produce physiological responses having therapeutic or performance-enhancing effects. For example, the visual code may be the product of iterations of the systems and methods disclosed herein to generate visual codes for particular neural responses or the visual code may be the product of other types of systems and methods. In particular implementations, the neural response may be one that affects one or more of the following: an emotional state, a brain state, a physiological state, an interoceptive state, and a behavioral state. In some cases, displaying the visual code or codes to the group of participants may induce a reaction in at least one user of the group of participants which may, in turn, result in one or more of the following: an emotional change, a physiological change, an interoceptive change, and a behavioral change. Furthermore, the induced reaction may result in one or more of the following: enhanced alertness, reduced anxiety, reduced pain, reduced depression, migraine relief, fear relief, and increased happiness.

As noted above, the visual code or codes may be based at least in part on a candidate visual code which is iteratively generated based on measured brain state and/or brain activity data. For example, the candidate visual code may be generated based at least in part on iterations in which the system receives a first set of brain state data and/or brain activity data measured while a participant is in a target state, e.g., a target emotional state. The first set of brain state data and/or brain activity data forms, in effect, a target for measured brain state/activity. With this point of reference, the candidate visual code is displayed to the participant while the participant is in a current state, i.e., a state other than the target state. The system receives a second set of brain state data and/or brain activity data measured during the displaying of the candidate visual code while the participant is in the current state. Based at least in part on a determined effectiveness of the candidate visual code, as described in further detail below, the system outputs the candidate visual code to be used as the visual stimulus or perturbs the candidate visual code and performs a further iteration.

The user devices also include, or are configured to communicate with, sensors to perform various types of physiological and brain state and activity measurements. This allows the system to receive feedback data indicative of responses of a user, or group of participants, during display of the visual codes to the users. The system performs analysis of the received feedback data indicative of the responses to produce various statistics and parameters, such as parameters indicative of a generalizable effect of the visual codes with respect to the neurological and/or physiological responses having therapeutic effects in users (or group of participants) and—by extension—other users who have not participated in such testing.

In particular implementations, the received feedback data may be obtained from a wearable device, e.g., a fitness band/watch, having sensors to measure physiological characteristics of the group of participants. The received feedback data may include one or more of the following: electrocardiogram (EKG) measurement data, pulse rate data, galvanic skin response, and blood pressure data. Furthermore, human behavioral responses may be obtained using video and/or audio monitoring, such as, for example, blinking, gaze focusing, and posture/gestures. In some cases, the received feedback data includes data characterizing one or more of the following: an emotional state, a brain state, a physiological state, an interoceptive state, and a behavioral state.

In particular implementations, the system may obtain physiological data, and other forms of characterizing data, from a group of participants to determine a respective baseline state of each user. The obtained physiological data may be used by the system to normalize the received feedback data from the group of participants based at least in part on the respective determined baseline state of each user. In some cases, the determined baseline states of the users may be used to, in effect, remediate a state in which the user is not able to provide high quality feedback data, such as, for example, if a user is in a depressed, inattentive, or agitated state. This may be done by providing known stimulus or stimuli to a particular user to induce a modified baseline state in the user. The known stimulus or stimuli may take various forms, such as visual, video, sound, sensory, chemical, and physical forms of stimulus.

Based on the parameters (e.g., parameters indicative of the generalizable effect of the visual codes) and/or statistics resulting from the analysis of the user feedback data for particular visual codes, a selection may be made as to whether to use the particular visual codes as the visual stimulus (e.g., as in the methods to provide a visual stimulus described herein) or to perform further iterations. For example, the selection may be based at least in part on comparing a parameter indicative of the generalizable effect of the visual code to defined criteria. In some cases, the parameter indicative of the generalizable effect of the visual code may be based at least in part on a measure of commonality of the neural responses among the group of participants. For example, the parameter indicative of the generalizable effect of the visual code may represent a percentage of users of the group of participants who meet one or more defined criteria for neural responses.

In the case of performing further iterations, the system may perform various mathematical operations on the visual codes, such as perturbing the visual codes and repeating the displaying of the visual codes, the receiving of the feedback data, and the analyzing of the received feedback data indicative of the responses of the group of participants to produce, inter alia, parameters indicative of the generalizable effect of the visual codes. In particular implementations, the perturbing of the visual codes may be performed using a machine learning model, a neural network, a convolutional neural network, a deep feedforward artificial neural network, an adversarial neural network, and/or an ensemble of neural networks. In some cases, the perturbing of the visual codes may be performed using an adversarial machine learning model which is trained to avoid representational images and/or semantic content to encourage generalizability and avoid cultural or personal bias.

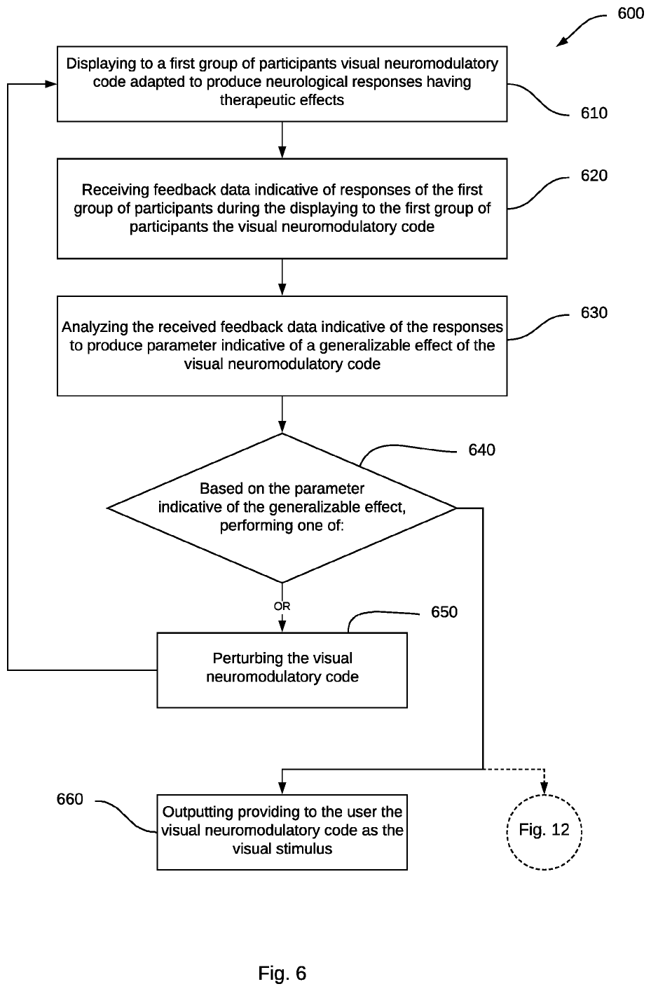

FIG. 6 depicts an embodiment of a method 600 to generate and provide to a user a visual stimulus to produce physiological responses having therapeutic or performance-enhancing effects. The disclosed method 600 is usable in a system such as that shown in FIG. 5, which is described above.

The method 600 includes displaying to a first group of participants (using one or more electronic displays) at least one visual code, at least one visual code adapted to produce physiological responses having therapeutic or performance-enhancing effects (610). The method 600 further includes receiving feedback data indicative of responses of the first group of participants during the displaying to the first group of participants the at least one visual code (620). The method 600 further includes analyzing the received feedback data indicative of the responses to produce at least one parameter indicative of a generalizable effect of the at least one visual code with respect to the neurological responses having therapeutic or performance-enhancing effects in participants of the first group of participants (630).

Based at least in part on the at least one parameter indicative of the generalizable effect of the at least one visual code, the method further includes performing one of: (i) outputting the at least one visual code as the visual stimulus, and (ii) perturbing the at least one visual code and repeating the displaying of the at least one visual code, the receiving the feedback data, and the analyzing the received feedback data indicative of the responses of the first group of participants to produce the at least one parameter indicative of the generalizable effect.

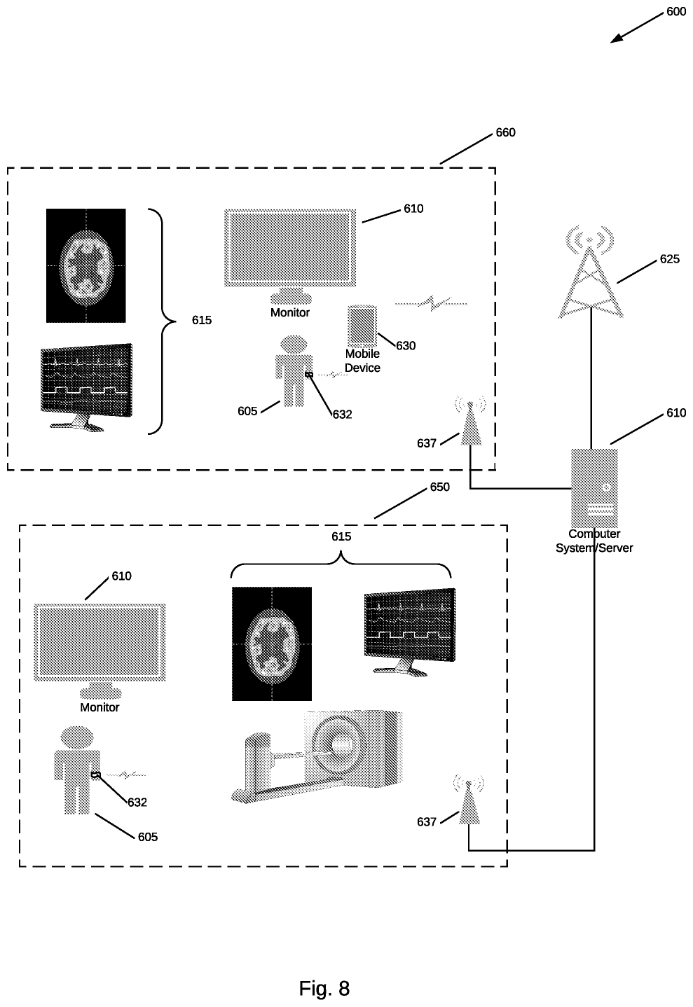

FIG. 8 depicts an embodiment of a system 600 to generate a visual stimulus, using brain state data and/or brain activity data measured while visual codes are displayed to a participant 605 in a target state and a current state, to produce physiological responses having therapeutic or performance-enhancing effects. The system 600 is processor-based and may include a network-connected computer system/server 610, or other type of computer system, having at least one processor and memory/storage. The memory/storage stores processor-executable instructions and data which, when executed by the at least one processor, cause the at least one processor to perform the necessary functions for the system to generate and provide to the user the visual stimulus.

In particular implementations, the computer system/server 610 is connected via a network 625 to a number of personal electronic devices 630, such as mobile phones and tablets, and computer systems. In some cases, the server may be connected via a network to one or more electronic displays which allow for viewing of visual neuromodulatory codes by users in a facility set up for individual and/or group testing, e.g., as discussed above with respect to FIGS. 5 and 6. A visual code may be generated based on feedback from one or more users and used as the visual stimulus to produce physiological responses having therapeutic or performance-enhancing effects, as discussed above.

The system 600 receives a first set of brain state data and/or brain activity data measured, e.g., using a first test set up 650 including a display 610 and various types of brain state and/or brain activity measurement equipment 615, while a test participant 605 is in a target state, e.g., a target emotional state. For example, the target state may be one in which the participant experiences enhanced alertness, reduced anxiety, reduced pain, reduced depression, migraine relief, fear relief, increased happiness, and/or various other positive, desirable states and/or various cognitive functions. The first set of brain state/activity data, thus, serves as a reference against which other measured sets of brain/activity can be compared to assess the effectiveness of a particular visual stimulus in achieving a desired state. The brain state data and/or brain activity data may include, inter alia, data acquired from one or more of the following: electroencephalogram (EEG), quantitative EEG, magnetoencephalography (MEG), single-photon emission computed tomography (SPECT), positron emission tomography (PET), functional magnetic resonance imaging (fMRI), and functional near-infrared spectroscopy (fNIRS)— measured while the participant is present in a facility equipped to make such measurements (e.g., a facility equipped with the first test set up 650). Various other types of physiological and/or neurological measurements may be used. Measurements of this type may be done in conjunction with an induced target state, as the participant will likely be present in the facility for a limited time.

The target state may be induced in the participant 605 by providing known stimulus or stimuli, which may be in the form of visual neuromodulatory codes, as discussed above, and/or various other forms of stimulus, e.g., visual, video, sound, sensory, chemical, and physical, etc. Alternatively, the target state may be achieved in the participant 605 by monitoring naturally occurring states, e.g., emotional states, experienced by the participant over a defined time period (e.g., a day, week, month, etc.) in which the participant is likely to experience a variety of emotional states. In such a case, the system 600 receives data indicative of one or more states (e.g., brain, emotional, cognitive, etc.) of the participant 605 and detects when the participant 605 is in the defined target state.

The system further displays to the participant 605, using an electronic display 610, a candidate visual code while the participant 605 is in a current state, the current state being different than the target state. For example, the participant 605 may be experiencing depression in a current state, as opposed to reduced depression and/or increased happiness in the target state. In particular implementations, the candidate visual code may be based at least in part on or more initial visual codes which are iteratively generated based at least in part on received feedback data indicative of responses of a group of participants during displaying of the one or more initial visual codes to the group of participants, as discussed above with respect to FIGS. 5 and 6.

The system 600 receives a second set of brain state data and/or brain activity data measured, e.g., using a second test set up 660 including a display 610 and various types of brain state and/or brain activity measurement equipment 615, during the display of the candidate visual code to the participant 605. As above, the brain state data and/or brain activity data may include, inter alia, data acquired from one or more of the following: electroencephalogram (EEG), quantitative EEG, magnetoencephalography (MEG), single-photon emission computed tomography (SPECT), positron emission tomography (PET), functional magnetic resonance imaging (fMRI), and functional near-infrared spectroscopy (fNIRS). It should be noted that psychiatric symptoms are produced by the patient's perception and subjective experience. Nevertheless, this does not preclude attempts to identify, describe, and correctly quantify this symptomatology using, for example, psychometric measures, cognitive and neuropsychological tests, symptom rating scales, various laboratory measures, such as, neuroendocrine assays, evoked potentials, sleep studies, brain imaging, etc. The brain imaging may include functional imaging (see examples above) and/or structural imaging, e.g., MRI, etc. In particular implementations, both the first and the second sets of brain state data and/or brain activity data may be obtained using the same test set up, i.e., either the first test set up 650 or the second test set up 660.

The system 600 performs an analysis the first set of brain state/activity data, i.e., the target state data, and the second set of brain state/activity data to produce at least one parameter indicative of an effectiveness of the candidate visual code with respect to the participant 605. For example, the participant 605 may provide feedback, such as survey responses and/or qualitative state indications using a personal electronic device 630, during the target state (i.e., the desired state) and during the current state. In addition, various types of measured feedback data may be obtained (i.e., in addition to the imaging data mentioned above) while the participant 605 is in the target and/or current state, such as electrocardiogram (EKG) measurement data, pulse rate data, blood pressure data, etc. The received feedback data may be obtained from a scale, an electronic questionnaire and a wearable device 632, e.g., a fitness band/watch, having sensors to measure physiological characteristics of the group of participant and communication features to communicate with the system 600, e.g., via a wireless link 637. Analysis of such information can provide parameters and/or statistics indicative of an effectiveness of the candidate visual code with respect to the participant.

Based at least in part on the parameters and/or statistics indicative of the effectiveness of the candidate visual code, the system 600 outputs the candidate visual code as the visual stimulus or performs a further iteration. In the latter case, the candidate visual code is perturbed (i.e., algorithmically modified, adjusted, adapted, randomized, etc.). In particular implementations, the perturbing of the candidate visual code may be performed using a machine learning model, a neural network, a convolutional neural network, a deep feedforward artificial neural network, an adversarial neural network, and/or an ensemble of neural networks. The displaying of the candidate visual code to the participant is repeated and the system receives a further set of brain state/activity data measured during the displaying of the candidate visual code. Analysis is again performed to determine whether to output candidate visual code as the visual stimulus or to perform a further iteration.

In particular implementations, the system may generate a candidate visual code from a set of “base” visual codes. In such a case, the system iteratively generates base visual codes having randomized characteristics, such as texture, color, geometry, etc. Neural responses to the base visual codes are obtained and analyzed. For example, the codes may be displayed to a group of participants with feedback data such as electrocardiogram (EKG) measurement data, pulse rate data, blood pressure data, etc., being obtained. As a further example, the codes may be displayed to participants with feedback data such as electroencephalogram (EEG) data, functional magnetic resonance imaging (fMRI) data, and magnetoencephalography (MEG) data being obtained. Based at least in part on the result of the analysis of the neural responses to the base visual codes, the system outputs a base visual code as the candidate visual code or perturbs one or more of the base visual codes and performs a further iteration. In particular implementations, the perturbing of the base visual codes may be performed using at is performed using at least one of: a machine learning model, a neural network, a convolutional neural network, a deep feedforward artificial neural network, an adversarial neural network, and an ensemble of neural networks.

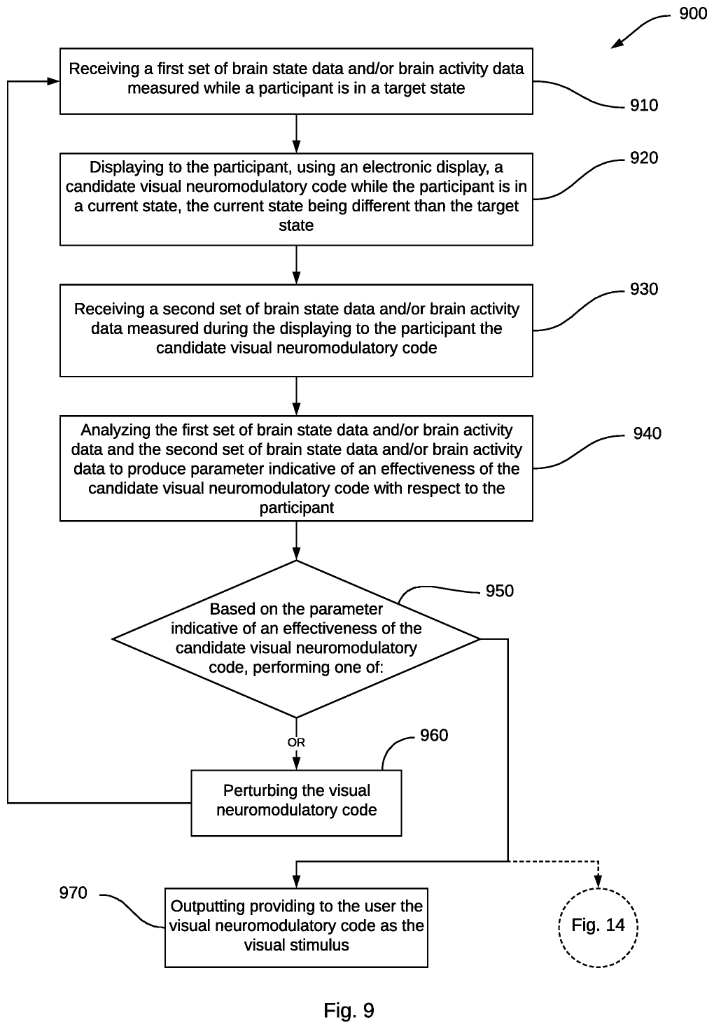

FIG. 9 depicts an embodiment of a method 900 to generate and provide to a user a visual stimulus to produce physiological responses having therapeutic or performance-enhancing effects. The disclosed method is usable in a system such as that shown in FIG. 8, which is described above.

The method 900 includes receiving a first set of brain state data and/or brain activity data measured while a participant is in a target state (910). The method 900 further includes displaying to the participant (using an electronic display) a candidate visual code while the participant is in a current state, the current state being different than the target state (920). The method 900 further includes receiving a second set of brain state data and/or brain activity data measured during the displaying to the participant the candidate visual code (930). The method 900 further includes analyzing the first set of brain state data and/or brain activity data and the second set of brain state data and/or brain activity data to produce at least one parameter indicative of an effectiveness of the candidate visual code with respect to the participant (940).

Based at least in part on the at least one parameter indicative of an effectiveness of the candidate visual code, the method further includes performing (950) one of: (i) outputting the candidate visual code as the visual stimulus (970), and (ii) perturbing the candidate visual code and repeating the displaying to the participant the candidate visual code, the receiving the second set of brain state data and/or brain activity data measured during the displaying to the participant the candidate visual code, and the analyzing the first set of brain state data and/or brain activity data and the second set of brain state data and/or brain activity data (960).

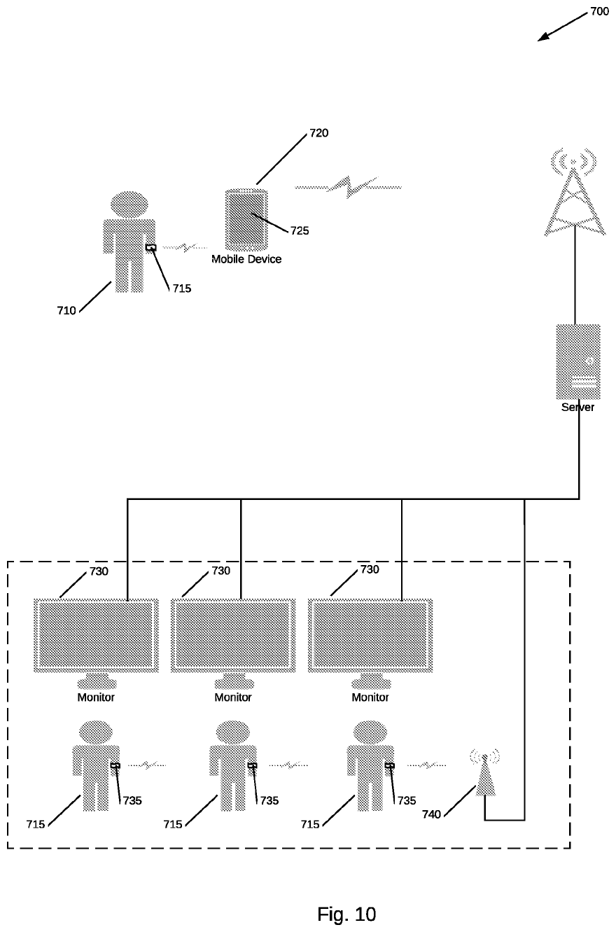

FIG. 10 depicts an embodiment of a 700 system to deliver a visual stimulus to a user 710, generated using visual codes displayed to a group of participants 715, to produce physiological responses having therapeutic or performance-enhancing effects. The system 700 is processor-based and may include a network-connected personal electronic device, e.g., a mobile device 720, or other type of network-connected user device (e.g., tablet, desktop computer, etc.), having and electronic display and at least one processor and memory/storage. The memory/storage stores processor-executable instructions and data which, when executed by the at least one processor, cause the at least one processor to perform the necessary functions for the system to provide the visual stimulus.

The system 700 outputs a visual code or codes to the electronic display 725 of the personal electronic device, e.g., mobile device 720. The visual codes are adapted to act as the visual stimulus to produce physiological responses having therapeutic or performance-enhancing effects. In particular implementations, the neural response may be one that affects an emotional state, a brain state, a physiological state, an interoceptive state, and/or a behavioral state of the user. The outputting to the electronic display 725, e.g., to the electronic display of the user's mobile device 720 (or other type of personal electronic device), the visual code or codes induces a reaction in the user resulting, for example, in an emotional change, a physiological change, an interoceptive change, and/or a behavioral change. The change in state and/or induced reaction in the user 710 may result in, inter alia, enhanced alertness, reduced anxiety, reduced pain, reduced depression, migraine relief, fear relief, and increased happiness. In implementations, the therapeutic effect may be usable as a substitute for, or adjunct to, anesthesia.

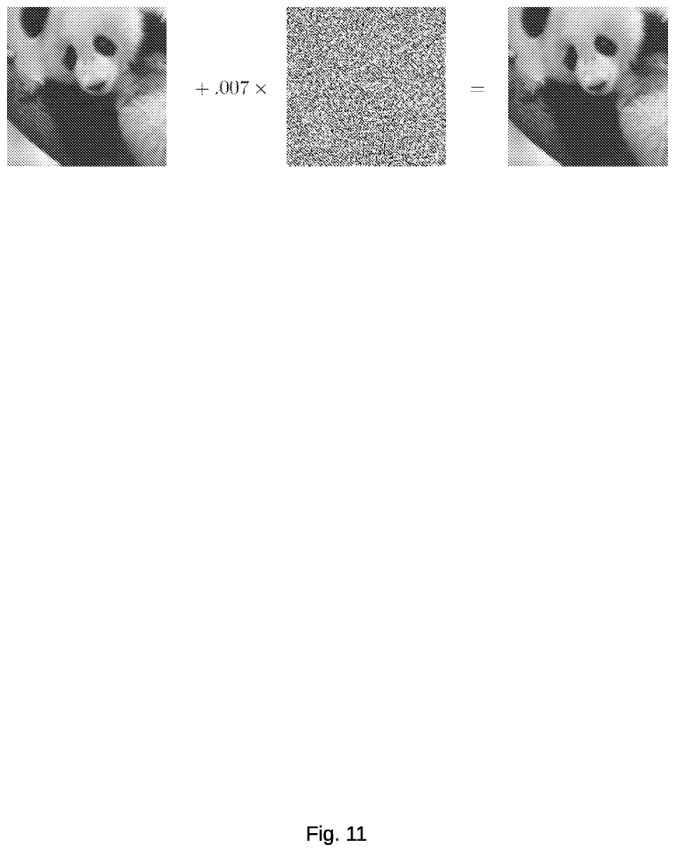

There are various methods of delivery for the visual neuromodulatory codes, including running in the background, “focused delivery” (e.g., user focuses on stimulus for a determined time with full attention), and overlaid—additive (e.g., a largely translucent layer overlaid on video or web browser content). For example, FIG. 11 depicts formation of a visual stimulus by overlaying a visual code (e.g., a non-semantic visual code) on content displayable on an electronic device. In such an implementation, the visual code overlaid on the displayable content may make a screen of the electronic device appear to be noisier, but a user generally would not notice the content of a visual code presented in this manner.

The visual codes are generated by iteratively performing a method such as the method described above with respect to FIGS. 5 and 6. In such a case, the method includes displaying to a group of participants 715 at least one test visual code, the at least one test visual code being adapted to activate the neural response to produce physiological responses having therapeutic or performance-enhancing effects.

The method further includes receiving feedback data indicative of responses of the group of participants 715 during the simultaneous displaying (e.g., using one or more electronic displays 730) to the group of participants 715 the at least one test visual code. The received feedback data may be obtained from a biomedical sensor, such as a wearable device 735 (e.g., smart glasses, watches, fitness bands/watches, wristbands, running shoes, rings, armbands, belts, helmets, buttons, etc.) having sensors to measure physiological characteristics of the participants 715 and communication features to communicate with the system 700, e.g., via a wireless link 740.

In general, biomedical sensors are electronic devices that transduce biomedical signals indicative of human physiology, e.g., brain waves and heat beats, into measurable electrical signals. Biomedical sensors can be divided into three categories depending on the type of human physiological information to be detected: physical, chemical, and biological. Physical sensors quantify physical phenomena such as motion, force, pressure, temperature, and electric voltages and currents—they are used to measure and monitor physiologic properties such as physical blood pressure, respiration, pulse, body temperature, heart sound, respiratory rate, blood viscosity, flow rate, flow rate, etc. Chemical sensors are utilized to measure chemical parameters such as oxygen and carbon dioxide concentration in the human metabolism, pH value, and ion levels in bodily fluids (e.g., Na⁺, K⁺, Ca²⁺, and Cl⁻). Biological sensors (i.e., “biosensors”) are used to detect biological parameters, such as tissues, cells, enzymes, antigens, antibodies, receptors, hormones, cholic acid, acetylcholine, serotonin, DNA and RNA, and other proteins and biomarkers.

The method further includes analyzing the received feedback data indicative of the responses to produce at least one parameter indicative of a generalizable effect of the at least one visual code with respect to the neurological responses having therapeutic effects in participants of the first group of participants. Based at least in part on the at least one parameter indicative of the generalizable effect of the at least one visual code, the method further includes performing one of: (i) outputting the at least one test visual code as the at least one visual code, and (ii) perturbing the at least one test visual code and performing a further iteration.

Referring again to FIG. 10, the system 700 obtains user feedback data indicative of responses of the user 710 during the outputting of the visual codes to the electronic display 725 of the mobile device 720. In particular implementations, the user feedback data may be obtained from sensors and/or user input. For example, the mobile device 720 may be wirelessly connected to a wearable device 740, e.g., a fitness band or watch, having sensors which measure physiological conditions of the user 710. The obtained user feedback data may include data characterizing an emotional state, a brain state, a physiological state, an interoceptive state, and/or a behavioral state of the user. Furthermore, the obtained user feedback data may include electrocardiogram (EKG) measurement data, pulse rate data, blood pressure data, etc.

In particular implementations, the system 700 may analyze the obtained user feedback data indicative of the responses of the user 710 to produce one or more parameters indicative of an effectiveness of the visual code or codes. In such a case, the system would iteratively perform (based at least in part on the at least one parameter indicative of the effectiveness of the at least one visual code) one of: (i) maintaining the visual code or codes as the visual stimulus, and (ii) perturbing the visual code or codes and performing a further iteration.

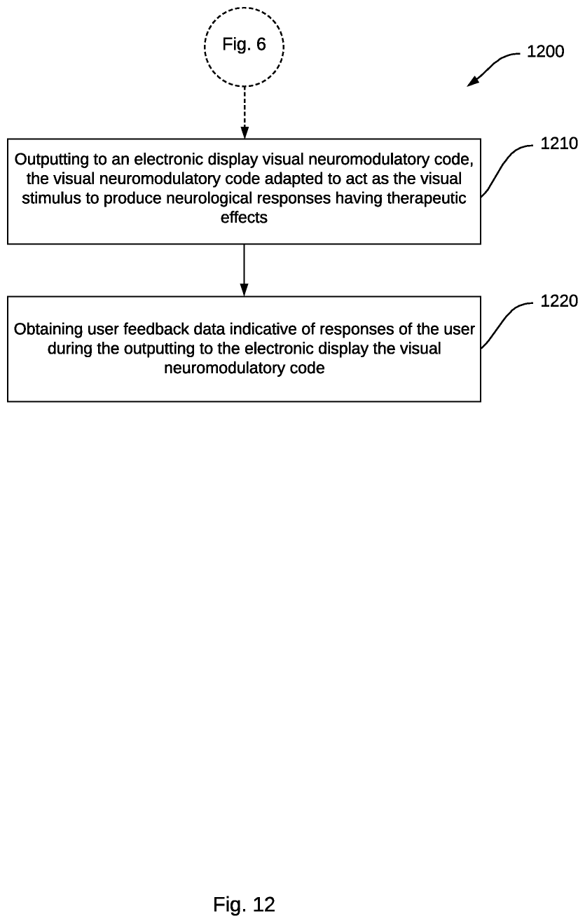

FIG. 12 depicts an embodiment of a method 1200 to deliver (i.e., provide) a visual stimulus to produce physiological responses having therapeutic or performance-enhancing effects. The disclosed method is usable in a system such as that shown in FIG. 10, which is described above. The method 1200 includes outputting to an electronic display of an electronic device at least one visual code, the at least one visual code adapted to act as the visual stimulus to produce physiological responses having therapeutic or performance-enhancing effects (1210). The method further includes obtaining user feedback data indicative of responses of the user during the outputting to the electronic display the at least one visual code (1220). In implementations, the at least one visual code may be generated using, for example, the method to generate a visual stimulus to produce physiological responses having therapeutic or performance-enhancing effects of FIG. 6, discussed above.

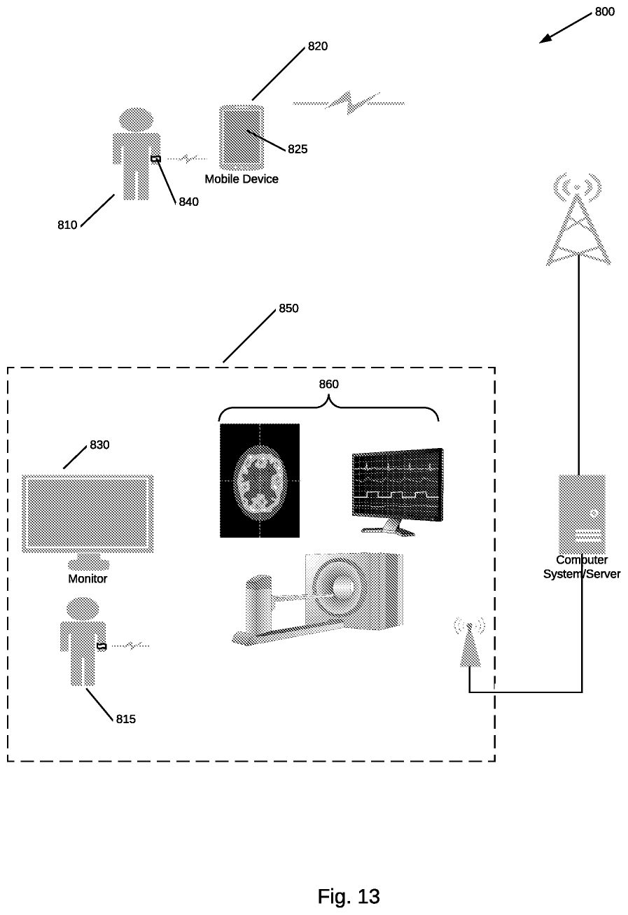

FIG. 13 depicts an embodiment of a system 800 to deliver a visual stimulus to a user 810, generated using brain state data and/or brain activity data measured while visual codes are displayed to a participant in a target state and a current state, to produce physiological responses having therapeutic or performance-enhancing effects. The system 800 is processor-based and may include a network-connected personal electronic device, e.g., a mobile device 820, or other type of network-connected user device (e.g., tablet, desktop computer, etc.), having and electronic display and at least one processor and memory/storage. The memory/storage stores processor-executable instructions and data which, when executed by the at least one processor, cause the at least one processor to perform the necessary functions for the system to provide the visual stimulus.

The system 800 outputs a visual code or codes to the electronic display 825 of the personal electronic device, e.g., mobile device 820. The visual codes are adapted to act as the visual stimulus to produce physiological responses having therapeutic or performance-enhancing effects. In particular implementations, the neural response may be one that affects an emotional state, a brain state, a physiological state, an interoceptive state, and/or a behavioral state of the user. The outputting to the electronic display 825, e.g., to the electronic display of the user's mobile device 820 (or other type of personal electronic device), the visual code or codes induces a reaction in the user resulting, for example, in an emotional change, a physiological change, an interoceptive change, and/or a behavioral change. The change in state and/or induced reaction in the user 810 may result in, inter alia, enhanced alertness, reduced anxiety, reduced pain, reduced depression, migraine relief, fear relief, and increased happiness.

The visual codes are generated by iteratively performing a method such as the method described above with respect to FIGS. 8 and 9. In such a case, the method includes receiving a first set of brain state data and/or brain activity data measured, e.g., using a test set up 850 including a display 830 and various types of brain state and/or brain activity measurement equipment 860, while a participant 815 is in a target state. The method further includes displaying to the participant 815 a candidate visual code (e.g., using one or more electronic displays 830) while the participant 815 is in a current state, the current state being different than the target state. The method further includes receiving a second set of brain state data and/or brain activity data measured, e.g., using the depicted test set up 850 (or a similar test set up), during the displaying to the participant 815 of the candidate visual code. The first set of brain state data and/or brain activity data and the second set of brain state data and/or brain activity data are analyzed to produce at least one parameter indicative of an effectiveness of the candidate visual code with respect to the participant. Based at least in part on the at least one parameter indicative of an effectiveness of the candidate visual code, the method further includes performing one of: (i) outputting the candidate visual code as the visual code, and (ii) perturbing the candidate visual code and performing a further iteration.

The system 800 obtains user feedback data indicative of responses of the user 810 during the outputting of the visual code or codes to the electronic display 825 of the user's mobile device 820. In particular implementations, the user feedback data may be obtained from sensors and/or user input. For example, the mobile device 820 may be wirelessly connected to a wearable device 840, e.g., a fitness band or watch, having sensors which measure physiological conditions of the user 810. The obtained user feedback data may include, inter alia, data characterizing an emotional state, a brain state, a physiological state, an interoceptive state, and a behavioral state. The obtained user feedback data may include, inter alia, electrocardiogram (EKG) measurement data, pulse rate data, and blood pressure data.

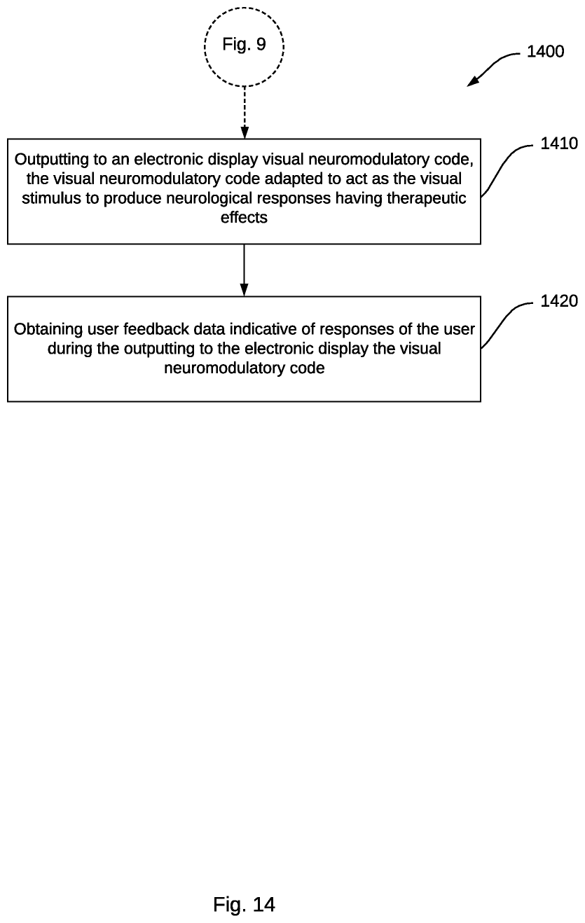

FIG. 14 depicts an embodiment of a method 1400 to deliver (i.e., provide) a visual stimulus to produce physiological responses having therapeutic or performance-enhancing effects. The disclosed method 1400 is usable in a system such as that shown in FIG. 13, which is described above. The method 1400 includes outputting to an electronic display at least one visual code, the at least one visual code adapted to act as the visual stimulus to produce physiological responses having therapeutic or performance-enhancing effects (1410). The method 1400 further includes obtaining user feedback data indicative of responses of the user during the outputting to the electronic display the at least one visual code (1420). In implementations, the at least one visual code may be generated using, for example, the method to generate a visual stimulus to produce physiological responses having therapeutic or performance-enhancing effects of FIG. 9, discussed above.

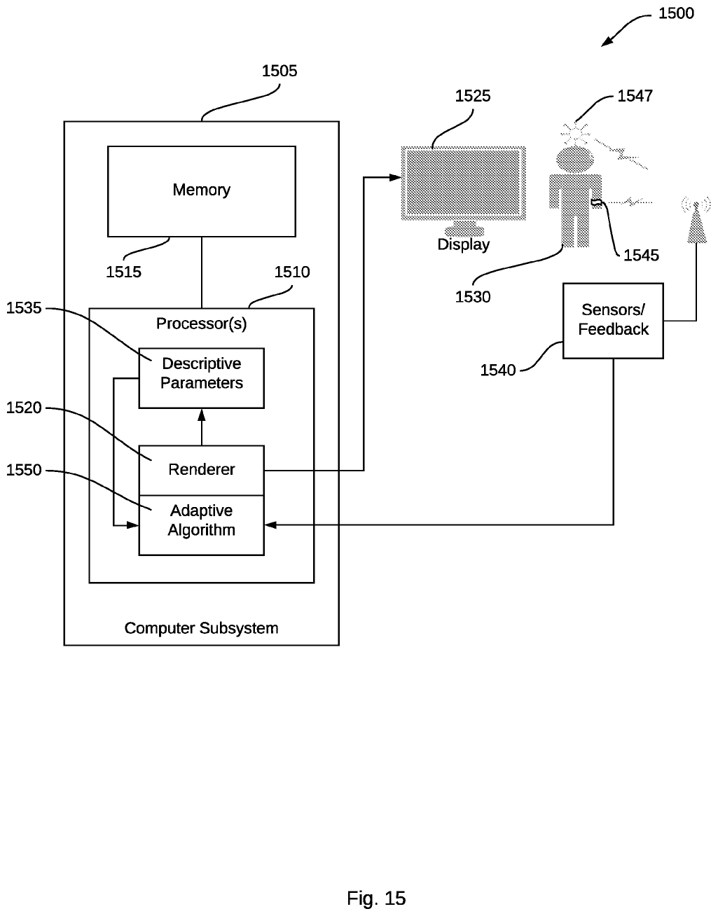

FIG. 15 depicts an embodiment of a system 1500 to generate visual neuromodulatory codes with a closed-loop approach using an optimized descriptive space to produce physiological responses having therapeutic or performance-enhancing effects. The system 1500 includes a computer subsystem 1505 comprising at least one processor 1510 and memory 1515 (e.g., non-transitory processor-readable medium). The memory 1515 stores processor-executable instructions which, when executed by the at least one processor 1510, cause the at least one processor 1510 to perform a method to generate the visual neuromodulatory codes. Specific aspects of the method performed by the processor 1510 are depicted as elements (e.g., code, software modules, and/or processes) within the processor for purposes of discussion only.

The renderer 1520 performs a rendering process to produce images (e.g., sequences of images) to be displayed on the display 1525 by generating video data based on specific inputs. In implementations, the output of the rendering process is a digital image stored as an array of pixels. Each pixel value may be a single scalar component or a vector containing a separate scalar value for each color component. The renderer 1520 may produce (i.e., synthesize) one or more visual neuromodulatory codes (e.g., a sequence of visual neuromodulatory codes) based on an initial set of rendering parameters (i.e., synthesis parameters) stored in the memory 1515. The video data and/or signal resulting from the rendering is output by the computer subsystem 1505 to the display 1525.

The system 1500 is configured to present the visual neuromodulatory codes to at least one subject 1530 by arranging the display 1525 so that it can be viewed by the subject 1530. For example, a video monitor may be provided in a location where it can be accessed by the subject 1530, e.g., a location where other components of the system are located. Alternatively, the video data may be transmitted via a network to be displayed on a video monitor or mobile device of the subject (not shown). In implementations, the subject may be one of the users of the system. In implementations, the visual neuromodulatory codes may be presented to a plurality of subjects, as described with respect to FIGS. 1-4.

In implementations, the system 1500 may present on the display 1525 a dynamic visual neuromodulatory code based on visual neuromodulatory codes. For example, a dynamic visual neuromodulatory code may be formed by combining a number of visual neuromodulatory codes to form a sequence of visual neuromodulatory codes. In a further example, a dynamic visual neuromodulatory code may be adapted to produce at least one of the following effects: a pulsating effect, a zooming effect, a flickering effect, and a color-shift effect. In some cases, the formation of the dynamic visual neuromodulatory code may include processing a set, e.g., a sequence, of visual neuromodulatory codes to produce intermediate images in the sequence of visual neuromodulatory codes. Various techniques, such as interpolation of pixels and gaussian averaging, may be used to produce the intermediate images.

In addition to outputting the visual neuromodulatory codes to the display 1525, the computer subsystem 1505 also includes a descriptive parameters calculator 1535 (e.g., code, a module, and/or a process) which computes values for descriptive parameters in a defined set of descriptive parameters characterizing the visual neuromodulatory codes produced by the renderer. In implementations, the defined set of descriptive parameters used to characterize the visual neuromodulatory codes is selected from a number of candidate sets of descriptive parameters by: rendering visual neuromodulatory codes; computing values of the descriptive parameters of each of the candidate sets of descriptive parameters; and modeling the performance of each of the candidate sets of descriptive parameters. Based on the modeled performance, one of the candidate sets of descriptive parameters is selected and used in the closed-loop process.

In some cases, the selected set of descriptive parameters comprises low-level statistics of visual neuromodulatory codes, including color, motion, brightness, and/or contrast. Another set of descriptive parameters may comprise metrics characterizing visual content of the visual neuromodulatory codes, including spatial frequencies and/or scene complexity. Another set of descriptive parameters may comprise intermediate representations of visual content of the visual neuromodulatory codes, in which case the intermediate representations may be produced by processing the visual neuromodulatory codes using a convolutional neural network trained to perform object recognition and encoding of visual information.

The system 1500 includes one or more sensors 1540, such as biomedical sensors, to measure physiological responses of the subject while the visual neuromodulatory codes are being presented to the subject 1530. For example, the system may include a wristband 1545 and a head-worn apparatus 1547 and may also include various other types of physiological and neurological feedback devices. In general, biomedical sensors include physical sensors, chemical sensors, and biological sensors. Physical sensors may be used to measure and monitor physiologic properties such as, for example, physical blood pressure, respiration, pulse, body temperature, heart sound, respiratory rate, blood viscosity, flow rate, flow rate, etc. Chemical sensors may be utilized to measure chemical parameters, such as, for example, oxygen and carbon dioxide concentration in the human metabolism, pH value, and ion levels in bodily fluids. Biological sensors (i.e., “biosensors”) are used to detect biological parameters, such as tissues, cells, enzymes, antigens, antibodies, receptors, hormones, cholic acid, acetylcholine, serotonin, DNA and RNA, and other proteins and biomarkers.

As noted above, the sensors 1540 used in the system 1500 may include wearable devices, such as, for example, wristbands 1545 and head-worn apparatuses 1547. Other examples of wearable devices include smart glasses, watches, fitness bands/watches, running shoes, rings, armbands, belts, helmets, buttons, etc. In implementations, the physiological responses of the subject may be measured using sensors adapted to measure, inter alia, one of the following: neurological responses, physiological responses, and behavioral responses. The sensors 1540 may include one or more of the following: EEG, MEG, fMRI, ECG, EMG, electrocardiogram, pulse rate, and blood pressure.

The computer subsystem 1505 receives and processes the physiological responses of the subject 1530 measured by the sensors 1540. Specifically, the measured physiological responses and the computed descriptive parameters (of the selected set of descriptive parameters) are input to an algorithm, e.g., an adaptive algorithm 1550, to produce adapted rendering parameters. The system 1500 iteratively repeats the rendering (e.g., by the renderer 1520), computing of descriptive parameters (e.g., by the descriptive parameters calculator 1535), presenting the visual neuromodulatory codes to the subject (e.g., by the display 1525), and processing (e.g., by the adaptive algorithm 1550), using the adapted rendering parameters, until the physiological responses of the subject meet defined criteria. In each iteration, the system 1500 generates one or more adapted visual neuromodulatory codes based on the adapted rendering parameters.

In implementations, the processing of the measured physiological responses of the subject is performed in real time with respect to presenting the visual neuromodulatory codes to a subject while measuring physiological responses of the subject. Alternatively, the processing of the measured physiological responses of the subject may be performed asynchronously with respect to presenting the visual neuromodulatory codes. For example, the measured physiological response data may be stored and processed in batches.

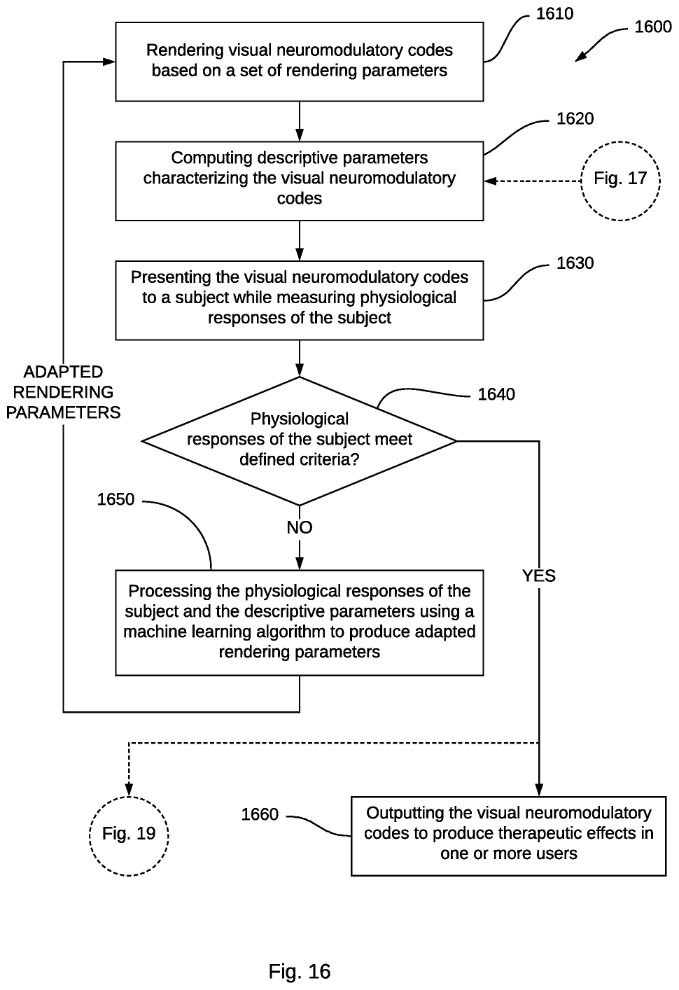

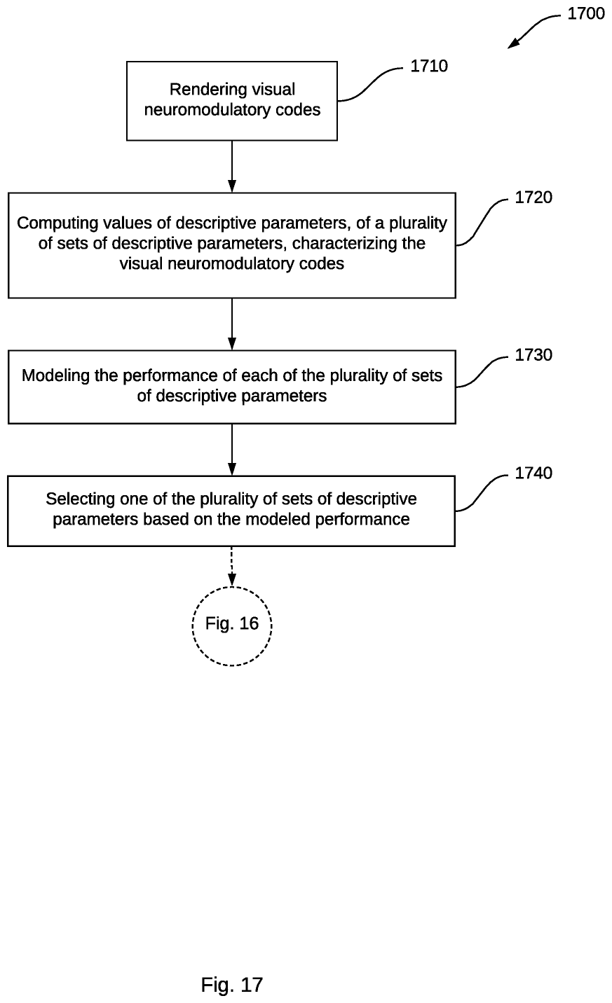

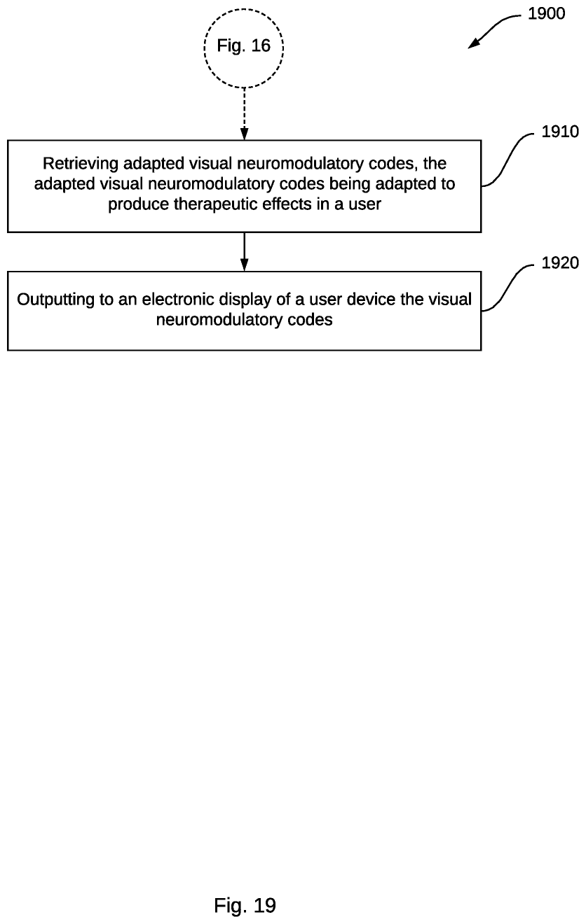

FIG. 16 depicts an embodiment of a method 1600, usable with the system of FIG. 15, to generate visual neuromodulatory codes with closed-loop approach using an optimized descriptive space. The method 1600 includes rendering visual neuromodulatory codes based on a set of rendering parameters (1610). A set of descriptive parameters is computed characterizing the visual neuromodulatory codes (1620). In implementations, the set of descriptive parameters may be the result of a method to determine a set of optimized descriptive parameters (see, e.g., FIG. 17 and related discussion below). The visual neuromodulatory codes are presented to a subject while measuring physiological responses of the subject (1630). A determination is made as to whether the physiological responses of the subject meet defined criteria (1640). If it is determined that the physiological responses of the subject do not meet the defined criteria, then the physiological responses of the subject and the set of descriptive parameters are processed using a machine learning algorithm to produce adapted rendering parameters (1650). The rendering (1610), the computing (1620), the presenting (1630), and the determining (1640) are repeated using the adapted rendering parameters. If, on the other hand, it is determined that the physiological responses of the subject meet the defined criteria, then the one or more adapted visual neuromodulatory codes are output to be used in producing physiological responses having therapeutic or performance-enhancing effects (1660). For example, in implementations, the adapted visual neuromodulatory codes may be used in a method to deliver visual neuromodulatory codes (see, e.g., FIG. 19 and related description below).

FIG. 17 depicts an embodiment of a method 1700 to determine an optimized descriptive space to characterize visual neuromodulatory codes. The method 1700 includes rendering visual neuromodulatory codes (1710). Values of descriptive parameters (of a plurality of sets of descriptive parameters) are computed characterizing the visual neuromodulatory codes (1720). The performance of each of the sets of descriptive parameters is modeled (1730). One of the sets of descriptive parameters is selected based on the modeled performance (1740).

FIG. 18 depicts an embodiment of a system 1800 to deliver visual neuromodulatory codes generated with closed-loop approach using an optimized descriptive space. The system 1800 includes an electronic device, referred to herein as a user device 1810, such as mobile device (e.g., mobile phone or tablet) or a virtual reality headset. When symptoms arise, a patient views the visual neuromodulatory codes on a user device, e.g., a smartphone or tablet, using an app or by streaming from a website. In disclosed embodiments, the app or web-based software may provide for the therapeutic visual neuromodulatory codes to be merged with (e.g., overlaid on) content being displayed on the screen, e.g., a website being displayed by a browser, a user interface of an app, or the user interface of the device itself, without interfering with normal use of such content. Audible stimuli may also be produced by the user device in conjunction, or separately from, the visual neuromodulatory codes.

In disclosed embodiments, the system may be adapted to personalize the visual neuromodulatory codes through the use of sensors and data from the user device (e.g., smartphone). For example, the user device may provide for measurement of voice stress levels based on speech received via a microphone of the user device, using an app or browser-based software and, in some cases, accessing a server and/or remote web services. The user device may also detect movement based on data from an accelerometer of the device. Eye-tracking, and pupil dilation measurement, may be performed using a camera of the user device. Furthermore, the user device may present questionnaires to a patent, developed using artificial intelligence, to automatically individualize the visual neuromodulatory codes and exposure time for optimal therapeutic effect. For enhanced effect, patients may opt to use a small neurofeedback wearable to permit further personalization of the visual neuromodulatory codes.

The user device 1810 comprises at least one processor 1815 and memory 1420 (e.g., random access memory, read-only memory, flash memory, etc.). The memory 1820 includes a non-transitory processor-readable medium adapted to store processor-executable instructions which, when executed by the processor 1815, cause the processor 1815 to perform a method to deliver the visual neuromodulatory codes. The user device 1810 has an electronic display 1825 adapted to display images rendered and output by the processor 1815.

The user device 1810 also has a network interface 1830, which may be implemented as a hardware and/or software-based component, including wireless network communication capability, e.g., Wi-Fi or cellular network. The network interface 1830 is used to retrieve one or more adapted visual neuromodulatory codes, which are adapted to produce physiological responses having therapeutic or performance-enhancing effects 1835. In some cases, visual neuromodulatory codes may be retrieved in advance and stored in the memory 1820 of the user device 1810.

In implementations, the retrieval, e.g., via the network interface 1830, of the adapted visual neuromodulatory codes may include communication via a network, e.g., a wireless network 1840, with a server 1845 which is configured as a computing platform having one or more processors, and memory to store data and program instructions to be executed by the one or more processors (the internal components of the server are not shown). The server 1845, like the user device 1810, includes a network interface, which may be implemented as a hardware and/or software-based component, such as a network interface controller or card (NIC), a local area network (LAN) adapter, or a physical network interface, etc. In implementations, the server 1845 may provide a user interface for interacting with and controlling the retrieval of the visual neuromodulatory codes.

The processor 1815 outputs, to the display 1825, visual neuromodulatory codes adapted to produce physiological responses having therapeutic or performance-enhancing effects in a user 1835 viewing the display 1825. The visual neuromodulatory codes may be generated by any of the methods disclosed herein. In this manner, the visual neuromodulatory codes are presented to the user 1835 so that the therapeutic or performance-enhancing effects can be realized. In outputting the adapted visual neuromodulatory codes to the display 1825 of the user device 1810, each displayed visual neuromodulatory code, or sequence of visual neuromodulatory codes (i.e., visual neuromodulatory codes displayed in a determined order), may be displayed for a determined time. These features provide, in effect, the capability of establishing a “dose” which can be prescribed for the user on an individualized basis, in a manner analogous to a prescription medication. In implementations, the determined display time of the adapted visual neuromodulatory codes may be adapted based on user feedback data indicative of responses of the user 1835. In implementations, outputting the adapted visual neuromodulatory codes may include overlaying the visual neuromodulatory codes on displayed content, such as, for example, the displayed output of an app running on the user device, the displayed output of a browser running on the user device 1810, and the user interface of the user device 1810.

The user device 1810 also has a near-field communication interface 1850, e.g., Bluetooth, to communicate with devices in the vicinity of the user device 1810, such as, for example, sensors (e.g., 1860), such as biomedical sensors, to measure physiological responses of the subject 1835 while the visual neuromodulatory codes are being presented to the subject 1835. In implementations, the sensors (e.g., 1860) may include wearable devices such as, for example, a wristband 1860 or head-worn apparatus (not shown). In implementations, the sensors may include components of the user device 1810 itself, which may obtain feedback data by, e.g., measuring voice stress levels, detecting movement, tracking eye movement, and receiving input to displayed prompts.

FIG. 19 depicts an embodiment of a method 1900, usable with the system of FIG. 18, to deliver visual neuromodulatory codes generated with closed-loop approach using an optimized descriptive space. The method 1900 includes retrieving adapted visual neuromodulatory codes, which are adapted to produce physiological responses having therapeutic or performance-enhancing effects (1910). The method 1900 further includes outputting to an electronic display of a user device the adapted visual neuromodulatory codes (1920). In implementations, the one or more adapted visual neuromodulatory codes may be generated, for example, according to the method of FIG. 16, discussed above.

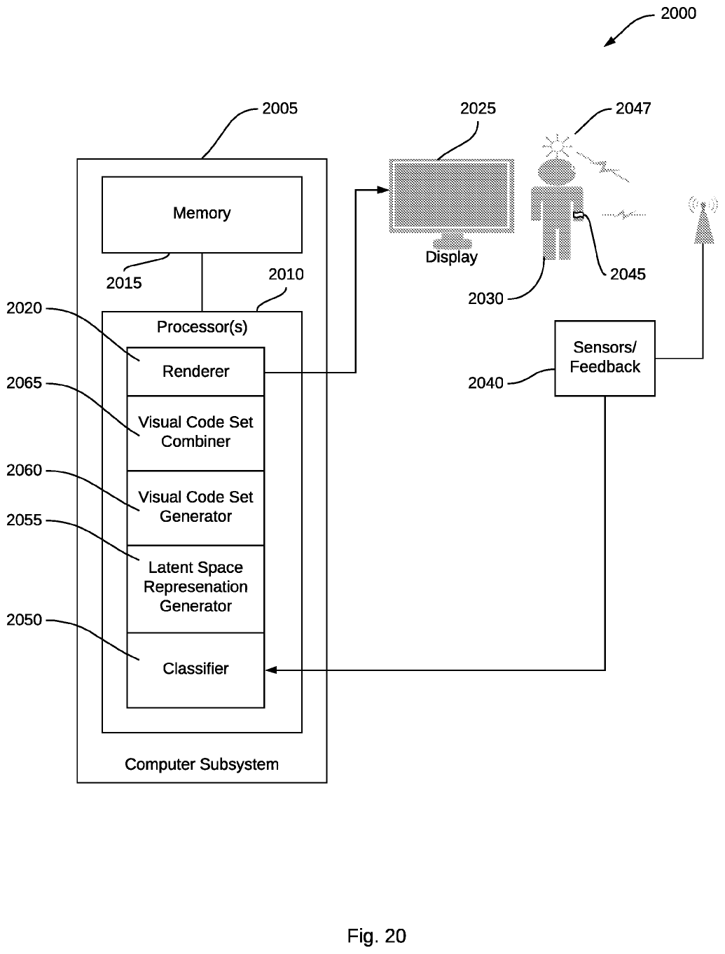

FIG. 20 depicts an embodiment of a system 2000 to generate visual neuromodulatory codes by reverse correlation and stimuli classification. The system 2000 includes a computer subsystem 2005 comprising at least one processor 2010 and memory 2015 (e.g., non-transitory processor-readable medium). The memory 2015 stores processor-executable instructions which, when executed by the at least one processor 2010, cause the at least one processor 2010 to perform a method to generate the visual neuromodulatory codes. Specific aspects of the method performed by the processor are depicted as elements (e.g., code, software modules, and/or processes) within the processor for purposes of discussion only.

The renderer 2020 produces images (e.g., sequences of images) to be displayed on the display 2025 by generating video data based on specific inputs. For example, the renderer 2020 may produce one or more visual neuromodulatory codes (e.g., a sequence of visual neuromodulatory codes) based on an initial set of rendering parameters stored in the memory 2015. The video data and/or signal resulting from the rendering is output by the computer subsystem 2005 to the display 2025.

The system 2000 is configured to present the visual neuromodulatory codes to a subject 2030 by, for example, displaying the visual neuromodulatory codes on a display 2025 arranged so that it can be viewed by the subject 2030. For example, a video monitor may be provided in a location where it can be accessed by the subject 2030, e.g., a location where other components of the system are located. Alternatively, the video data may be transmitted via a network to be displayed on a video monitor or mobile device of the subject. In implementations, the subject 2030 may be one of the users of the system.

In implementations, the system 2000 may present on the display 2025 a dynamic visual neuromodulatory code based on visual neuromodulatory codes. For example, a dynamic visual neuromodulatory code may be formed by combining a number of visual neuromodulatory codes to form a sequence of visual neuromodulatory codes. In a further example, a dynamic visual neuromodulatory code may be adapted to produce at least one of the following effects: a pulsating effect, a zooming effect, a flickering effect, and a color-shift effect. In some cases, the formation of the dynamic visual neuromodulatory code may include processing a set, e.g., a sequence, of visual neuromodulatory codes to produce intermediate images in the sequence of visual neuromodulatory codes. Various techniques, such as interpolation of pixels and gaussian averaging, may be used to produce the intermediate images.

The system 2000 includes one or more sensors 2040, such as biomedical sensors, to measure physiological responses of the subject while the visual neuromodulatory codes are being presented to the subject 2030. For example, the system may include a wristband 2045 and a head-worn apparatus 2047 and may also include various other types of physiological and neurological feedback devices. Other examples of wearable devices include smart glasses, watches, fitness bands/watches, running shoes, rings, armbands, belts, helmets, buttons, etc. In implementations, the physiological responses of the subject may be measured using sensors adapted to measure, inter alia, one of the following: neurological responses, physiological responses, and behavioral responses. The sensors 2040 may include one or more of the following: EEG, MEG, fMRI, ECG, EMG, electrocardiogram, pulse rate, and blood pressure.

The computer subsystem 2005 receives and processes feedback data from the sensors 2040, e.g., the measured physiological responses of the subject 2030. For example, a classifier 2050 receives feedback data while a first set of visual neuromodulatory codes is presented to a subject 2030 and classifies the first set of visual neuromodulatory codes into classes based on the physiological responses of the subject 2030 measured by the sensors 2040. A latent space representation generator 2055 is configured to generate a latent space representation (e.g., using a convolutional neural network) of visual neuromodulatory codes in at least one specified class. A visual neuromodulatory code set generator 2060 is configured to generate a second set of visual neuromodulatory codes based on the latent space representation of the visual neuromodulatory codes in the specified class. A visual neuromodulatory code set combiner 2065 is configured to incorporate the second set of visual neuromodulatory codes into a third set of visual neuromodulatory codes.

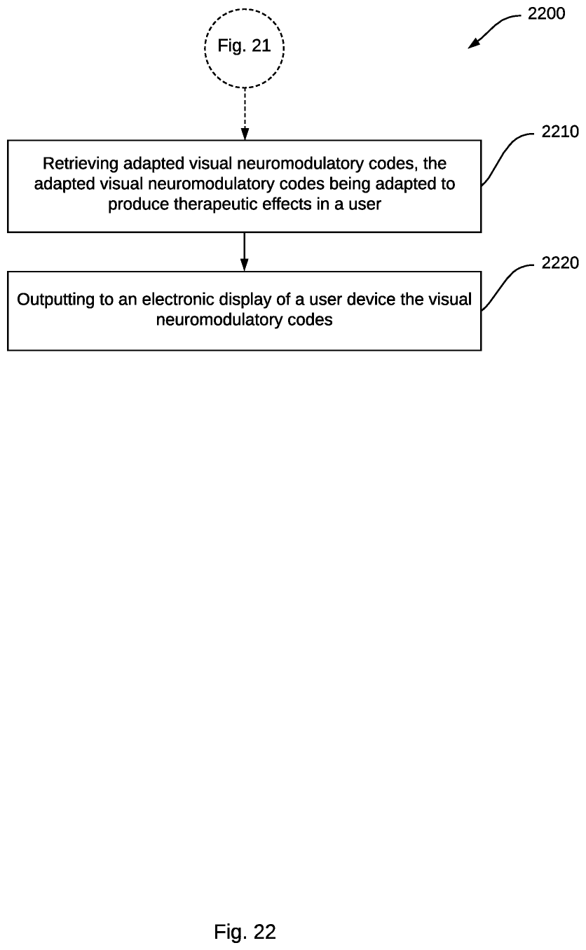

The system 2000 iteratively repeats, using the third set of visual neuromodulatory codes, the classifying the visual neuromodulatory codes, generating the latent space representation, generating the second set of visual neuromodulatory codes, and the combining until a defined condition is achieved. Specifically, the iterations continue until a change in the latent space representation of the visual neuromodulatory codes in specified class, from one iteration to a next iteration, meets defined criteria. The system then outputs the third set of visual neuromodulatory codes to be used in producing physiological responses having therapeutic or performance-enhancing effects. For example, in implementations, the adapted visual neuromodulatory codes may be used in a method to deliver visual neuromodulatory codes (see, e.g., FIG. 22 and related description below). In implementations, the subject 2030 may be one of the users of the system.

In implementations, at least a portion of the first set of visual neuromodulatory codes may be generated randomly. Furthermore, the classifying of the first set of visual neuromodulatory codes into classes based on the measured physiological responses of the subject may include detecting irregularities in the time domain and/or time-frequency domain of the measured physiological responses of the subject 2040.

In implementations, the processing of the measured physiological responses of the subject is performed in real time with respect to presenting the visual neuromodulatory codes to a subject while measuring physiological responses of the subject. Alternatively, the processing of the measured physiological responses of the subject may be performed asynchronously with respect to presenting the visual neuromodulatory codes. For example, the measured physiological response data may be stored and processed in batches.

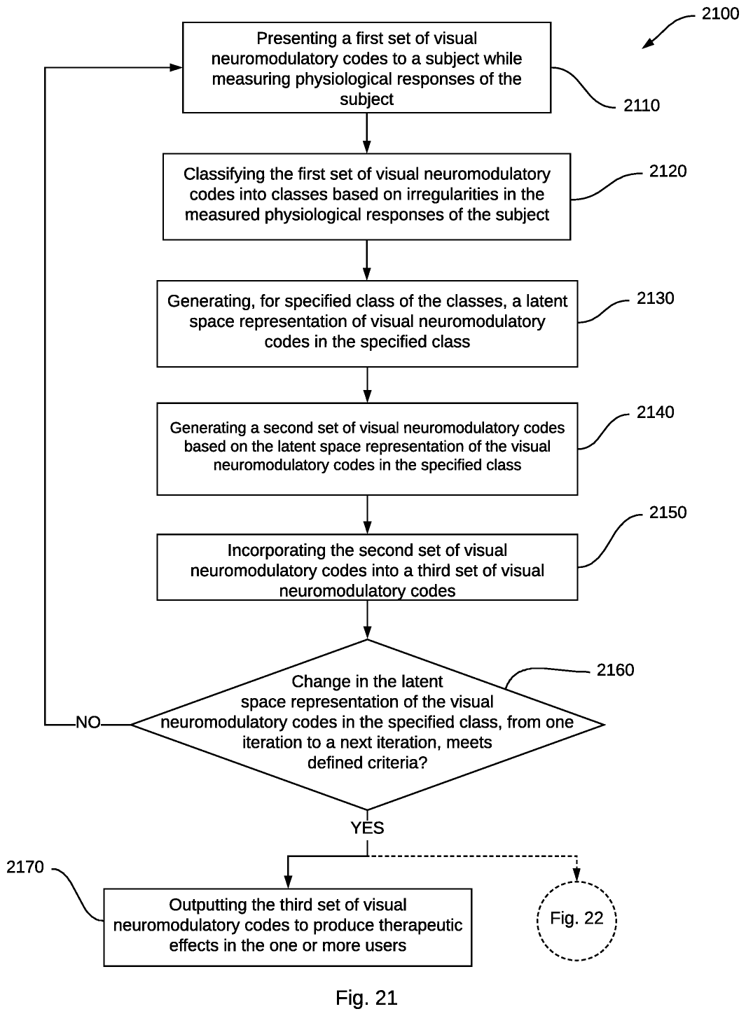

FIG. 21 depicts an embodiment of a method 2100, usable with the system of FIG. 20 to generate visual neuromodulatory codes by reverse correlation and stimuli classification. The method 2100 includes presenting a first set of visual neuromodulatory codes to a subject while measuring physiological responses of the subject (2110). The first set of visual neuromodulatory codes is classified into classes based on the measured physiological responses of the subject (2120). For at least one specified class of the classes, a latent space representation is generated of visual neuromodulatory codes (2130). A second set of visual neuromodulatory codes is generated based on the latent space representation of the visual neuromodulatory codes in the specified class (2140). The second set of visual neuromodulatory codes is incorporated into a third set of visual neuromodulatory codes (2150). If it is determined that a change in the latent space representation of the visual neuromodulatory codes in the at least one specified class, from one iteration to a next iteration, does not meet defined criteria (2160), then the classifying the visual neuromodulatory codes (2120), generating the latent space representation (2130), generating the second set of visual neuromodulatory codes (2140), and the combining (2150) are iteratively repeated using the third set of visual neuromodulatory codes. If the change in the latent space representation of the visual neuromodulatory codes in the at least one specified class, from one iteration to a next iteration, is determined to meet defined criteria (2160), then the third set of visual neuromodulatory codes are output to be used in producing physiological responses having therapeutic or performance-enhancing effects (2170). In implementations, the third set of visual neuromodulatory codes may be used in a method to deliver visual neuromodulatory codes generated by reverse correlation and stimuli classification (see FIG. 22 and related description below).

FIG. 22 depicts an embodiment of a method 2200, usable with the system of FIG. 18, to deliver visual neuromodulatory codes generated by reverse correlation and stimuli classification. The method 2200 includes retrieving one or more adapted visual neuromodulatory codes, the one or more adapted visual neuromodulatory codes being adapted to produce physiological responses having therapeutic or performance-enhancing effects (2210). The method 2200 further includes outputting to an electronic display of a user device the one or more adapted visual neuromodulatory codes (2220). In implementations, the one or more adapted visual neuromodulatory codes may be generated, for example, according to the method of FIG. 21, discussed above.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. Those of skill in the art will recognize that many of the methods or algorithms set out herein may employ additional acts, may omit some acts, and/or may execute acts in a different order than specified. The various implementations described above can be combined to provide further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A method to generate non-figurative visual neuromodulatory codes adapted to produce physiological responses having therapeutic or performance-enhancing effects, the method comprising:
rendering a visual neuromodulatory code based on a set of rendering parameters;
outputting the visual neuromodulatory code to be displayed on a plurality of electronic screens to be viewed simultaneously by a plurality of subjects;
receiving output of one or more sensors that measure, during said outputting the visual neuromodulatory code, one or more physiological responses of each of the plurality of subjects;
calculating a value of an outcome function based on said one or more physiological responses of each of the plurality of subjects;
determining an updated predictive model based at least in part on a current predictive model and the calculated value of the outcome function, the predictive model providing an estimated value of the outcome function for a given set of rendering parameters;
calculating values for a set of adapted rendering parameters;
iteratively repeating the method using the set of adapted rendering parameters, to produce an adapted visual neuromodulatory code, until a defined set of stopping criteria are satisfied; and
outputting, upon satisfying the defined set of stopping criteria, the adapted visual neuromodulatory code based on the set of adapted rendering parameters.

2. The method of claim 1, wherein the outcome function is indicative of a therapeutic effectiveness of the visual neuromodulatory code.

3. The method of claim 2, wherein the outcome function is indicative of a degree of generalizability, among the plurality of subjects, of the therapeutic effectiveness of the visual neuromodulatory code.

4. The method of claim 1, wherein said rendering the visual neuromodulatory code based on the set of rendering parameters comprises projecting a latent representation of the visual neuromodulatory code onto a parameter space of a rendering engine.

5. The method of claim 1, wherein said calculating values for a set of adapted rendering parameters based at least in part on:
determining, using the updated predictive model, an estimated value of the outcome function for a plurality of values of the set of rendering parameters to form a response characteristic; and
determining values of the set of adapted rendering parameters based at least in part on the response characteristic.

6. The method of claim 5, wherein said determining values of the set of adapted rendering parameters comprises applying an acquisition function to the response characteristic to optimize selection of the values of the set of adapted rendering parameters.

7. The method of claim 1, further comprising:
characterizing a sample visual neuromodulatory code using a plurality of defined descriptive spaces, each including one or more descriptive parameters, said characterizing comprising analyzing the sample visual neuromodulatory code to determine values of the descriptive parameters of each of said plurality of defined descriptive spaces;
modeling performance of each of said plurality of defined descriptive spaces; and
selecting one of said plurality of defined descriptive spaces based at least in part on said modeling to define constituent parameters of the set of rendering parameters.

8. The method of claim 7, wherein said modeling of the performance of each of said plurality of defined descriptive spaces comprises using a Bayesian optimization algorithm.

9. The method of claim 7, wherein, a first descriptive space, of said plurality of defined descriptive spaces, comprises low-level statistics of said sample visual neuromodulatory code, including at least one of color, brightness, and contrast.

10. The method of claim 9, wherein a second descriptive space, of said plurality of defined descriptive spaces, comprises metrics characterizing visual content of said sample visual neuromodulatory code, including at least one of spatial frequencies and scene complexity.

11. The method of claim 10, wherein a third descriptive space, of said plurality of defined descriptive spaces, comprises intermediate representations of visual content of said sample visual neuromodulatory code, the intermediate representations produced by processing said sample visual neuromodulatory code using a convolutional neural network trained to perform object recognition and encoding of visual information.

12. The method of claim 1, wherein, in said receiving output of said one or more sensors, said one or more sensors are adapted to measure at least one of the following: neurological responses, physiological responses, and behavioral responses.

13. The method of claim 1, wherein said one or more sensors comprise one or more of the following: electroencephalogram (EEG), quantitative EEG, magnetoencephalography (MEG), single-photon emission computed tomography (SPECT), positron emission tomography (PET), functional magnetic resonance imaging (fMRI), functional near-infrared spectroscopy (fNIRS), EMG, electrocardiogram (ECG), pulse rate, blood pressure, and galvanic skin response (GSR).

14. The method of claim 1, further comprising:
repeating the method to produce a plurality of adapted visual neuromodulatory codes; and
forming a dynamic adapted visual neuromodulatory code based at least in part on said plurality of adapted visual neuromodulatory codes.

15. The method of claim 14, wherein said forming a dynamic adapted visual neuromodulatory code comprises combining said plurality of adapted visual neuromodulatory codes to form a sequence of adapted visual neuromodulatory codes.

16. The method of claim 15, wherein said forming a dynamic adapted visual neuromodulatory code further comprises processing said plurality of adapted visual neuromodulatory codes to form intermediate images in the sequence of adapted visual neuromodulatory codes.

17. The method of claim 1, wherein the stopping criteria are based on at least one of: a predefined number of iterations, characteristics of the acquisition function, and a determination that convergence of the outcome function with target criteria will not occur within a defined number of iterations.

18. (canceled)

19. A method to provide non-figurative visual neuromodulatory codes adapted to produce physiological responses having therapeutic or performance-enhancing effects, the method comprising:
retrieving one or more adapted visual neuromodulatory codes, said one or more adapted visual neuromodulatory codes being adapted to produce physiological responses having therapeutic or performance-enhancing effects; and
outputting to an electronic display of a device viewable by a user said one or more adapted visual neuromodulatory codes,
wherein said one or more adapted visual neuromodulatory codes are generated by performing
rendering a visual neuromodulatory code based on a set of rendering parameters;
outputting the visual neuromodulatory code to be displayed on a plurality of electronic screens to be viewed simultaneously by a plurality of subjects;
receiving output of one or more sensors that measure, during said outputting the visual neuromodulatory code, one or more physiological responses of each of the plurality of subjects;
calculating a value of an outcome function based on said one or more physiological responses of each of the plurality of subjects;
determining an updated predictive model based at least in part on a current predictive model and the calculated value of the outcome function, the predictive model providing an estimated value of the outcome function for a given set of rendering parameters;
calculating values for a set of adapted rendering parameters;
iteratively repeating the method using the set of adapted rendering parameters, to produce an adapted visual neuromodulatory code, until a defined set of stopping criteria are satisfied; and
outputting, upon satisfying the defined set of stopping criteria, the adapted visual neuromodulatory code based on the set of adapted rendering parameters.

20. The method of claim 19, wherein said retrieving said one or more adapted visual neuromodulatory codes comprises receiving said one or more adapted visual neuromodulatory codes via a network or retrieving said one or more adapted visual neuromodulatory codes from a memory of the user device.

21. The method of claim 19, wherein, in said outputting to the electronic display of the user device said one or more adapted visual neuromodulatory codes, each of said one or more adapted visual neuromodulatory codes is displayed for a determined time period, the determined time period being adapted based on user feedback data indicative of responses of the user.

22. The method of claim 19, wherein said outputting to the electronic display of the user device said one or more adapted visual neuromodulatory codes comprises combining said one or more adapted visual neuromodulatory codes with displayed content.

23. The method of claim 22, wherein the displayed content comprises at least one of: displayed output of an app, displayed output of a browser, and a user interface of the user device.

24. The method of claim 19, further comprising obtaining user feedback data indicative of responses of the user during said outputting to an electronic display of the user device said one or more adapted visual neuromodulatory codes.

25. The method of claim 24, wherein said obtaining user feedback data indicative of responses of the user comprises using components of the user device to perform at least one of: measuring voice stress levels, detecting movement, tracking eye movement, and receiving input to displayed prompts.

26. The method of claim 24, wherein said obtaining user feedback data indicative of responses of the user comprises receiving data from a wearable neurological sensor.

27. A system to generate non-figurative visual neuromodulatory codes adapted to produce physiological responses having therapeutic or performance-enhancing effects, the system comprising:
at least one processor; and
at least one non-transitory processor-readable medium that stores processor-executable instructions which, when executed by the at least one processor, cause the at least one processor to perform:
rendering a visual neuromodulatory code based on a set of rendering parameters;
outputting the visual neuromodulatory code to be displayed on a plurality of electronic screens to be viewed simultaneously by a plurality of subjects;
receiving output of one or more sensors that measure, during said outputting the visual neuromodulatory code, one or more physiological responses of each of the plurality of subjects;
calculating a value of an outcome function based on said one or more physiological responses of each of the plurality of subjects;
determining an updated predictive model based at least in part on a current predictive model and the calculated value of the outcome function, the predictive model providing an estimated value of the outcome function for a given set of rendering parameters;
calculating values for a set of adapted rendering parameters;
iteratively repeating the method using the set of adapted rendering parameters, to produce an adapted visual neuromodulatory code, until a defined set of stopping criteria are satisfied; and
outputting, upon satisfying the defined set of stopping criteria, the adapted visual neuromodulatory code based on the set of adapted rendering parameters.
